(19) United States
(12) Patent Application Publication
(10) Pub. No.: US 2010/0028447 A1
(43) Pub. Date: Feb. 4, 2010

(54) Intranasal, Buccal, And Sublingual Administration Of Metanicotine Analogs

(75) Inventors: Sharon Rae Letchworth, Kernersville, NC (US); Merouane Bencherif, Winston-Salem, NC (US); Gary Maurice Dull, Lewisville, NC (US); David Moore, Advance, NC (US); John W. James, Winston-Salem, NC (US)

Correspondence Address: Pepper Hamilton LLP, 400 Berwyn Park, 899 Cassatt Road, Berwyn, PA, 19312-1183, US

(73) Assignee: TARGACEPT, INC., Winston-Salem, NC (US)

(21) Appl. No.: 12/523,406

(22) PCT Filed: Jan. 22, 2008
(86) PCT No.: PCT/US2008/000802

§ 371 Date: Oct. 5, 2009

Related U.S. Application Data
(60) Provisional application No. 60/886,012, filed on Jan. 22, 2007.
(60) Provisional application No. 60/895,805, filed on Mar. 20, 2007.

(51) Int. Cl. A61K 31/4412; A61K 31/60; A61K 9/14; A61K 9/68; A61P 25/00; A61P 25/28; A61P 25/08; A61P 25/04; A61P 25/18; A61P 25/24

(52) U.S. Cl. 424/489; 514/351; 514/161; 424/48

(57) ABSTRACT

The present invention generally relates to pharmaceutical compositions for the intranasal, buccal, or sublingual administration of metanicotine analogs.

### BACKGROUND OF THE INVENTION

A variety of metanicotine analogs have been proposed for use in treating a variety of disorders, predominantly via oral administration. See, for example, U.S. Pat. No. 5,616,716, U.S. Pat. No. 5,861,423, U.S. Pat. No. 6,232,316, U.S. Pat. No. 6,958,399, and U.S. Pat. No. 7,045,538, the contents of which are hereby incorporated by reference with regard to such analogs. Some of these compounds, however, suffer from relatively fast degradation in vivo, which makes it difficult to administer them to the site of action via routes that involve first pass metabolism in the gut wall and liver. Even for metanicotine analogs that do not have rapid first pass metabolism, routes of administration other than the oral route may provide advantageous benefits, particularly if they provide improvements in therapeutic levels or the onset of activity.

Routes for administration alternative to oral are discussed in each of the following: Graff and Pollack, Journal of Pharmaceutical Sciences, volume 94 (2005), #6, page 1187; American Academy of Pediatrics Committee on Drugs, Pediatrics, volume 100, #1 (July, 1997), page 143;

Shojaei, Journal of Pharmacy and Pharmaceutical Science, volume 1, #1, page 15 (1998); Johnson and Quay, Expert Opinion on Drug Delivery, volume 2, # 2, page 281 (2005); Sheckler et al., Drug Delivery Technology, volume 6, # 5, page 56 (2006); Patent Application US 2006 0084656 A1; Leonard et al., Journal of Pharmaceutical Sciences, volume 94, #8, page 1736 (2005); and Smith et al., Neuropsychopharmacology, volume 31, page 637 (2006); each of which is incorporated herein for background.

The present invention provides novel compositions and methods for administering certain metanicotine analogs.

### BRIEF SUMMARY OF THE INVENTION

The present invention includes a composition of E-metanicotine, (2S)-(4E)-N-methyl-5-(3-(5-methoxypyridin)yl)-4-penten-2-amine, (2S)-(4E)-N-methyl-5-(3-(5-isopropoxypyridin)yl)-4-penten-2-amine, or a pharmaceutically acceptable salt thereof, along with a pharmaceutically acceptable carrier for intranasal, buccal, or sublingual administration.

In one embodiment the composition includes E-metanicotine or a pharmaceutically acceptable salt thereof. In another embodiment the composition includes (2S)-(4E)-N-methyl-5-(3-(5-methoxypyridin)yl)-4-penten-2-amine or a pharmaceutically acceptable salt thereof. In another embodiment the composition includes (2S)-(4E)-N-methyl-5-(3-(5-isopropoxypyridin)yl)-4-penten-2-amine or a pharmaceutically acceptable salt thereof.

In one embodiment, the composition of the present invention further includes an absorption promoting agent. In one embodiment, the composition of the present invention further includes one or more excipient, diluent, binder, lubricant, glidant, disintegrant, desensitizing agent, emulsifier, mucosal adhesive, solubilizer, suspension agent, viscosity modifier, ionic tonicity agent, buffer, carrier, surfactant, flavor, or mixture thereof.

In one embodiment, the composition of the present invention is a liquid, liquid spray, microspheres, semisolid, gel, or powder.

In one embodiment, the composition of the present invention is a solid dosage form for buccal or sublingual administration that disintegrates in an oral cavity at body temperature and optionally may adhere to the body tissue of the oral cavity. In an additional embodiment, the composition of the present invention further includes one or more excipient, diluent, binder, lubricant, glidant, disintegrant, desensitizing agent, emulsifier, mucosal adhesive, solubilizer, suspension agent, viscosity modifier, ionic tonicity agent, buffer, carrier, surfactant, flavor, or mixture thereof. In an additional embodiment the composition is formulated as a tablet, pill, bioadhesive patch, sponge, film, lozenge, hard candy, wafer, sphere, lollipop, disc-shaped structure, or spray.

Compounds, such as those of the present invention, which bind to neuronal nictonic acetylcholine specific receptor sites are useful in modulating cholinergic function. Accordingly, the compounds of the present invention are useful in the treatment of various conditions or disorders including, but not limited to, inflammatory bowel disease, including ulcerative colitis, pyoderma gangrenosum, and Crohn's disease, irritable bowel syndrome, spastic dystonia, pain, including acute pain, chronic pain, neurologic pain, neuropathic pain, female-specific pain, post-surgical pain, inflammatory pain, or cancer pain, celiac sprue, pouchitis, vasoconstriction, anxiety, including generalized anxiety disorder, panic disorder, depression, bipolar disorder, autism, Pick's disease, Creutzfeld-Jakob disease, multiple sclerosis, mania, sleep disorders, jet lag, amyotrophic lateral sclerosis (“ALS”), cognitive dysfunction, hypertension, bulimia, anorexia, obesity, cardiac arrhythmia, gastric acid hypersecretion, ulcer, pheochromocytoma, progressive supranuclear palsy, chemical dependencies and addictions, including dependencies on or addictions to nicotine or other tobacco product, alcohol, benzodiazepines, barbiturates, opioids, or cocaine, headache, migraine, stroke, traumatic brain injury, obsessive-compulsive disorder (“OCD”), psychosis, Huntington's chorea, tardive dyskinesia, hyperkinesias, dyslexia, schizophrenia, schizophreniform disorder, schizoaffective disorder, or cognitive deficits in schizophrenia, multi-infarct dementia, age-related cognitive decline, seizure, epilepsy, including petit mal absence epilepsy, age-associated memory impairment, mild cognitive impairment, pre-senile dementia, early onset Alzheimer's disease, senile dementia, senile dementia of the Alzheimer's type, Alzheimer's disease, Parkinson's disease, Lewy body dementia, HIV-dementia, vascular dementia, AIDS dementia complex, attention deficit disorder, attention deficit hyperactivity disorder, rage outburst, and Tourette's syndrome.

With regard to pain, the present invention includes a method for alleviating pain through administration to a subject in need thereof an effective amount of a composition of the present invention. In one embodiment, the type of pain is acute pain, chronic pain, neurologic pain, neuropathic pain, female-specific pain, post-surgical pain, inflammatory pain, or cancer pain.

With regard to central nervous system (“CNS”) disorders, the present invention includes a method for treating central nervous system disorders through administration to a subject in need thereof an effective amount of a composition of the present invention. In one embodiment, the central nervous system disorder is associated with an alteration in normal neurotransmitter release. In one embodiment, the central nervous system disorder is dyslexia, Parkinsonism, Parkinson's disease, Pick's disease, Huntington's chorea, tardive dyskinesia, hyperkinesia, progressive supranuclear palsy, Creutzfeld-Jakob disease, multiple sclerosis, amyotrophic lateral sclerosis, epilepsy, mania, anxiety, depression, panic disorders, bipolar disorders, generalized anxiety disorder, obsessive compulsive disorder, rage outbursts, Tourette's syndrome, autism, age-associated memory impairment, mild cognitive impairment, pre-senile dementia, early onset Alzheimer's disease, senile dementia, dementia of the Alzheimer's type, Lewy body dementia, HIV-dementia, vascular dementia, Alzheimer's disease, AIDS dementia complex, attention deficit disorder, attention deficit hyperactivity disorder, schizophrenia, schizophreniform disorder, schizoaffective disorder, or cognitive deficits in schizophrenia.

In one embodiment, the present invention includes (2S)-(4E)-N-methyl-5-(3-(5-isopropoxypyridin)yl)-4-penten-2-amine or a salt thereof, including but not limited to the hydroxybenzoic acid salt, with a pharmaceutically acceptable carrier for intranasal, buccal, or sublingual administration for use in the treatment or prophylaxis of one or more of mild to moderate dementia of Alzheimer's type, attention deficit disorder, attention deficit hyperactivity disorder, mild cognitive impairment, age associated memory impairment, schizophrenia, and cognitive deficit in schizophrenia. In one embodiment, present invention includes the use of (2S)-(4E)-N-methy-5-(3-(5-isopropoxypyridin)yl)-4-penten-2-amine or a salt thereof, including but not limited to the hydroxybenzoic acid salt, with a pharmaceutically acceptable carrier for intranasal, buccal, or sublingual administration in the manufacture of a medicament for use in the treatment or prophylaxis of one or more of mild to moderate dementia of Alzheimer's type, attention deficit disorder, attention deficit hyperactivity disorder, mild cognitive impairment, age associated memory impairment, schizophrenia, and cognitive deficit in schizophrenia.

In one embodiment, the present invention includes (2S)-(4E)-N-methyl-3-(5-methoxypyridin)yl)-4-penten-2-amine or a salt thereof, with a pharmaceutically acceptable carrier for intranasal, buccal, or sublingual administration for use in the treatment or prophylaxis of one or more of mild to moderate dementia of Alzheimer's type, attention deficit disorder, attention deficit hyperactivity disorder, mild cognitive impairment, age associated memory impairment, schizophrenia, and cognitive deficit in schizophrenia. In one embodiment, present invention includes the use of (2S)-(4E)-N-methyl-5-(3-(5-methoxypyridin)yl)-4-penten-2-amine or a salt thereof, with a pharmaceutically acceptable carrier for intranasal, buccal, or sublingual administration in the manufacture of a medicament for use in the treatment or prophylaxis of one or more of mild to moderate dementia of Alzheimer's type, attention deficit disorder, attention deficit hyperactivity disorder, mild cognitive impairment, age associated memory impairment, schizophrenia, and cognitive deficit in schizophrenia.

### BRIEF DESCRIPTION OF THE FIGURES

FIG. 1 illustrates permeability results for Compound B across cultured human respiratory epithelium (average±STD, with N=3). In the presence of the MAO inhibitor Pargyline, the permeation of Compound B significant increased as compared to the permeation of Compound B alone, the depicted * indicates p<0.05, two-tailed t-test.

FIG. 2 illustrates permeability results for a MAO Substrate Phenylethylamine (PEA) across cultured human respiratory epithelium (average±STD, with N=3). In the presence of the MAO inhibitor Pargyline, the permeation of PEA significant increased as compared to the permeation of PEA alone, the depicted * indicates p<0.05, two-tailed t-test.

FIG. 3 illustrates permeability results for Compound A across cultured human respiratory epithelium (average±STD, with N=3). As illustrated there is no significant difference between Compound A permeation when applied in the absence or presence of the CYP2D6 inhibitor Quinidine.

FIG. 4 illustrates permeability results for a CYP2D6 Substrate Bufuralol, across cultured human respiratory epithelium (average±STD, with N=3). As illustrated there is no significant difference in Bufuralol permeation when applied in the absence or presence of the CYP2D6 inhibitor Quinidine.

FIG. 5 illustrates the brain penetration of Compound B (average±STD, with N=3). As illustrated, in the presence of the MAO inhibitor Pargyline, the brain concentration of Compound B significantly increased compared to the brain concentration of Compound B alone, the * indicates p<0.05, two-tailed t-test.

FIG. 6 illustrates the brain penetration of Compound A (average±STD, with N=3). As illustrated, there is no significant difference between Compound A brain penetrations when perfused in the absence or presence of the CYP2D6 inhibitor Quinidine.

FIG. 7 illustrates the average brain concentrations (ng/g) of Compound C. As illustrated, at ten (10) minutes post-dose the brain levels for the 5 mg/kg doses were higher for the intranasal than that for the oral dosing.

FIG. 8 illustrates the average plasma concentrations (ng/mL) of Compound C. As illustrated, at ten (10) minutes post-dose the plasma levels for the 5 mg/kg doses were higher for the intranasal than that for the oral dosing.

FIG. 9 illustrates the average brain/plasma ratio [(ng/g)/(ng/mL)] of Compound C. The illustrated low values demonstrate that the integrity of the blood-brain barrier was maintained throughout the relevant portion of the study, as described herein in further detail.

### DETAILED DESCRIPTION OF THE INVENTION

E-metanicotine and its salts have relatively poor bioavailability when administered orally due to metabolism during the first pass in the liver. (2S)-(4E)-N-methyl-5-(3-(5-methoxypyridin)yl)-4-penten-2-amine and (2S)-(4E)-N-methyl-5-(3-(5-isopropoxypyridin)yl)-4-penten-2-amine and their respective salts have acceptable bioavailability when administered orally, but intranasal, buccal, or sublingual administration provides other advantages over oral administration.

As described in detail hereinbelow, some of the methods of the present invention involve treating or preventing disease or disorders affected through modulation of cholinergic function. As an example, central nervous system disorders include disorders characterized by dysfunction of nicotinic cholinergic neurotransmission, including disorders involving neuromodulation of neurotransmitter release, such as dopamine release. The central nervous system (CNS) disorders can be characterized by an alteration in normal neurotransmitter release. Other methods of the present invention involve treating certain other conditions, including but not limited to, alleviating pain and treating or preventing inflammation. Each of the methods of the present invention involve administering to a subject an effective amount of a composition of the present invention via an intranasal, buccal, or sublingual route to treat or prevent the disorder, including but not limited to the alleviation or elimination of pain or inflammation.

The compositions for intranasal, buccal, or sublingual administration include an effective amount of one or more metanicotine analogs or a pharmaceutically acceptable salt thereof, along with one or more pharmaceutically acceptable carrier or excipients. The compositions can be in the form of powders, dispersions, or solutions of the active compound. The compositions optionally can include components such as permeation enhancers, bioadhesive polymers, and means for providing instantaneous or modified release, such as sustained release, of the active ingredients. The compositions can also include one or more pharmaceutically acceptable flavoring or other taste-masking agent.

The pharmaceutical compositions include effective amounts of compounds E-metanicotine, (2S)-(4E)-N-methyl-5-(3-(5-isopropoxypyrdin)yl)-4-penten-2-amine, or (2S)-(4E)-N-methyl-5-(3-(5-methoxypyridin)yl)-4-penten-2-amine, or a combination thereof, to interact with relevant nicotinic receptor sites of a subject. The pharmaceutical compositions of the present invention provide therapeutic benefit to individuals suffering from such disorders and exhibiting clinical manifestations of such disorders in that the compounds within those compositions, when employed in effective amounts, have the potential to: (i) exhibit nicotinic pharmacology and affect relevant nicotinic receptor sites, including but not limited to, acting as a pharmacological agonist to activate nicotinic receptors; and (ii) elicit neurotransmitter secretion, and hence prevent and suppress the symptoms associated with those diseases. In addition, the compounds are expected to have the potential to: (i) increase the number of nicotinic cholinergic receptors of the brain of the patient; and (ii) exhibit neuroprotective effects, while exhibiting a preferred profile, namely not causing significant increases in blood pressure and heart rate, significant negative effects upon the gastrointestinal tract, nor significant effects upon skeletal muscle.

The term “intranasal delivery” or “nasal delivery” as used herein means a method for drug absorption through and within the nose. The term “buccal delivery” as used herein means a method for presenting the drug for absorption through the buccal, including inner cheek, tissue. The term “sublingual delivery” means delivery of the active agent under the tongue. Collectively, these are transmucosal delivery methods.

Drugs can be absorbed through mucosal surfaces, such as those in the nasal passage and in the oral cavity. Drug delivery via mucosal surfaces can be efficient because they lack the stratum corneum of the epidermis, a major barrier to absorption across the skin. Mucosal surfaces are also typically rich in blood supply, which can rapidly transport drugs systemically while avoiding significant degradation by first-pass hepatic metabolism.

There are three routes of absorption for drugs sprayed onto the olfactory mucosa, including by the olfactory neurons, by the supporting cells and surrounding capillary bed, and into the cerebro-spinal fluid. Absorption of drugs through the nasal mucosa tends to be rapid.

Like intranasal administration, oral transmucosal absorption is generally rapid because of the rich vascular supply to the mucosa and the lack of a stratum corneum in the epidermis. Such drug transport typically provides a rapid rise in blood concentrations, and similarly avoids the enterohepatic circulation and immediate destruction by gastric acid or partial first-pass effects of gut wall and hepatic metabolism.

Drugs typically need to have prolonged exposure to an oral mucosal surface for significant drug absorption to occur. Factors affecting drug delivery include taste, which can affect contact time, and drug ionization. Drug absorption is generally greater from the buccal or oral mucosa than from the tongue and gingiva. One limitation associated with buccal drug delivery is low flux, which often results in low drug bioavailability. Low flux may be somewhat offset by using buccal penetration enhancers, as are known in the art, to increase the flux of drugs through the mucosa.

In either of the intranasal or buccal routes, drug absorption can be delayed or prolonged, or uptake may be almost as rapid as if an intravenous bolus were administered. Because of the high permeability of the rich blood supply, the sublingual route can provide a rapid onset of action.

The intranasal, buccal, and sublingual routes can be effective in delivering E-metanicotine, which exhibits appropriate affinity and selectivity for, and activity at, a relevant receptor, but which is otherwise too rapidly metabolized in vivo, for example, by liver first pass metabolism, if delivered orally. These routes are also effective for delivering (2S)-(4E)-N-methyl-5-(3-(5-methoxypyridin)yl)-4-penten-2-amine and (2S)-(4E)-N-methyl-5-(3-(5-isopropoxypyridin)yl)-4-penten-2-amine, even though these compounds are not as quickly metabolized.

The intranasal, buccal, and sublingual routes can also be more effective than the oral route in that these routes can provide for relatively faster absorption and onset of therapeutic action. Further, the intranasal, buccal, and sublingual routes can be preferred for use in treating patients who have difficulty in swallowing tablets, capsules, or other oral solids, or those who have disease-compromised intestinal absorption. Accordingly, there are many advantages to the intranasal, buccal, or sublingual administration of E-metanicotine, (2S)-(4E)-N-methyl-5-(3-(5-methoxypyridin)yl)-4-penten-2-amine, (2S)-(4E)-N-methyl-5-(3-(5-isopropoxypyridin)yl)-penten-2-amine, or a pharmaceutically acceptable salt thereof (herein collectively referred to as active ingredients).

The phrase “active ingredient” means a compound E-metanicotine, (2S)-(4E)-N-methyl-5-(3-(5-isopropoxypyridin)yl)-4-penten-2-amine, or (2S)-(4E)-N-methyl-5-(3-(5 methoxypyridin)yl)-4-penten-2-amine. As used herein, “active ingredient” includes a prodrug of a compound. As used herein, “active ingredient” includes a pharmaceutically acceptable salt, hydrate, or solvate of a compound or a prodrug.

Salts encompassed within the term “pharmaceutically acceptable salts” refer to non-toxic salts of the compounds of this invention. Salts of the compounds of the present invention may comprise, but should not be limited to acid addition salts. Representative salts include acetate, aspartate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxybenzoate, hydroxynaphthoate, iodide, isethionate, lactate, lactobionate, laurate, lysine hydrochloride, malate, maleate, mandelate, mesylate, methylsulfate, monopotassium maleate, mucate (galactartrate)/hemimucate (hemigalactartrate), napsylate, nitrate, N-methylglucamine, orotate, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. One embodiment of the present invention includes a pharmaceutically acceptable salt formed through acid addition with tartaric acid, hydroxybenzoic acid, phosphoric acid, edisylic acid, citric acid, orotic acid, mandelic acid, sulfuric acid, 1,5-naphthalenedisulfonic acid, aspartic acid, and lysine monohydrochloride acid. Other salts, which are not pharmaceutically acceptable, may be useful in the preparation of compounds of this invention and these should be considered to form a further aspect of the invention. As a non-limiting example, an active ingredient of the present invention includes the hydroxybenzoic acid salt of (2S)-(4E)-N-methyl-5-(3-(5-isopropoxypyridin)yl)-4-penten-2-amine.

As used herein, the term “solvate” refers to a complex of variable stoichiometry formed by a solute, namely in this invention, a compound of Formulae herein described, or a salt or prodrug thereof, and a solvent. Such solvents, for the purpose of the invention, should not interfere with the biological activity of the solute. Non-limiting examples of suitable solvents include, but are not limited to water, methanol, ethanol, and acetic acid. Preferably, the solvent used is a pharmaceutically acceptable solvent. Non-limiting examples of suitable pharmaceutically acceptable solvents include water, ethanol, and acetic acid. Most preferably, the solvent used is water.

As used herein, a prodrug includes a biohydrolyzable ester or biohydrolyzable amide of a compound herein described.

The phrase “other ingredients” means any excipients, diluents, binders, lubricants, glidants, disintegrants, desensitizing agents, emulsifiers, mucosal adhesives, solubilizers, suspension agents, viscosity modifiers, ionic tonicity agents, buffers, carriers, surfactants, flavors, and mixtures thereof that are formulated with one or more active ingredient.

The phrases “appropriate period of time” or “suitable period of time” mean the period of time necessary to achieve a desired effect or result. For example, a mixture can be blended until a potency distribution is reached that is within an acceptable range for a given application or use of the blended mixture.

The phrases “unit dose,” “unit dosage,” or “unit dosage form” mean a physically discrete unit that contains a predetermined quantity of active ingredient calculated to produce a desired therapeutic effect. The dosage form can be in any suitable form for buccal, sublingual, or intranasal administration, which forms are well known to those of skill in the art.

The phrase “effective amount,” as used herein means the amount determined by such considerations as are known in the art for treating or preventing central nervous system disorders, or treating or preventing addiction, inflammation, or pain in an individual. The phrase includes providing measurable relief in treated individuals, such as through exhibiting improvements including but not limited to more rapid recovery, improvement of symptoms, elimination of symptoms, reduction of complications, or other measurements as appropriate and known to those skilled in the medical arts.

In any of the embodiments described herein, the active blend of a dosage form generally includes one or more other ingredient and will depend upon the purpose for which the active ingredient is being applied. In general, intranasal, buccal, and sublingual formulations are made of other ingredients including, but not limited to, excipients, diluents, binders, lubricants, glidants, disintegrants, desensitizing agents, emulsifiers, mucosal adhesives, solubilizers, suspension agents, viscosity modifiers, ionic tonicity agents, buffers, carriers, flavors and mixtures thereof.

### I. Metanicotine Compounds

The compounds that are the subject of the present invention include: (E)-metanicotine, (2S)-(4E)-N-methyl-5-(3-(5-isopropoxypyridin)yl)-4-penten-2-amine, and (2S)-(4E)-N-methyl-5-(3-(5-methoxypyridin)yl)-4-penten-2-amine, or a pharmaceutically acceptable salt thereof. The formulas for these compounds' free bases are shown below:

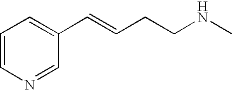

### (E)-metanicotine

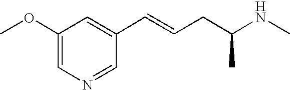

### (2S)-(4E)-N-methyl-5-(3-(5-methoxypyridin)yl)-4-penten-2-amine

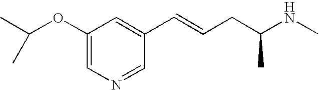

### (2S)-(4E)-N-methyl-5-(3-(5-isopropoxypyridin)yl)-4 penten-2-amine

### II. Compound Preparation

The synthesis of (E)-metanicotine is described by Ruecroft and Woods in U.S. Pat. No. 5,663,356, herein incorporated by reference with regard to such synthesis. The synthesis of salts of (E)-metanicotine can be accomplished by combining (E)-metanicotine with various inorganic and organic acids in appropriate solvents, as exemplified in U.S. Pat. No. 6,743,812 and PCT WO2006/053039, each herein incorporated by reference with regard to such synthesis.

The synthesis of (2S-(4E)-N-methyl-5-(3-(5-methoxypyridin)yl)-4-penten-2-amine and (2S)-(4E)-N-methyl-5-(3)-(5-isopropoxypyridin)yl)-4-penten-2-amine can be carried out, for example, using procedures described in U.S. Pat. Nos. 7,045,538 and 6,958,399, each herein incorporated by reference with regard to such synthesis. The synthesis of salts of (2S)-(4E)-N-methyl-5-(3-(5-methoxypyridin)yl)-4-penten-2-amine and (2S)-(4E)-N-methyl-5-(3-(5 isopropoxypyridin)yl)-4-penten-2-amine can be accomplished by combining (2S)-(4E)-N-methyl-5-(3-5-methoxypyridin)yl)-4-penten-2-amine or (2S)-(4E)-N-methyl-5-(3-(5-isopropoxypyridin)yl)-4-penten-2-amine with various inorganic and organic acids in appropriate solvents, as exemplified in U.S. Pat. No. 6,432,954, U.S. Pat. No. 7,045,538 and PCT WO/053039, each herein incorporated by reference with regard to such synthesis. The hemigalactarate salts of (E)-metanicotine, (2S)-(4E)-N-methyl-5-(3-(5-isopropoxypyridin)yl)-4-penten-2-amine, and (2S-(4E)-N-methyl-5-(3-(5-methoxypyridin)yl)-4-penten-2-amine can be prepared using the techniques set forth in U.S. Pat. No. 7,045,538 and U.S. Pat. No. 6,958,399, each herein incorporated by reference with regard to such synthesis.

### III. Intranasal Compositions

Relative to an oral dosage form such as a tablet or capsule, intranasal delivery provides for rapid absorption, faster onset of therapeutic action and avoidance of gut wall or liver first pass metabolism. For patients who have difficulty in swallowing tablets, capsules or other solids or those who have intestinal failure, the intranasal delivery route may be preferred.

The compositions for nasal administration include (E)-metanicotine, (2S)-(4E)-N-methyl-5-(3-(5 isopropoxypyridin)yl)-4-penten-2-amine, or (2S)-(4E)-N-methyl-5-(3-(5 methoxypyridin)yl)-4-penten-2-amine, or a pharmaceutically acceptable salt thereof, and optionally can also include other ingredients including, but not limited to, carriers and excipients, such as absorption-promoting agents which promote nasal absorption of the active ingredient after nasal administration. Other optional excipients include diluents, binders, lubricants, glidants, disintegrants, desensitizing agents, emulsifiers, mucosal adhesives, solubilizers, suspension agents, viscosity modifiers, ionic tonicity agents, buffers, carriers, flavors and mixtures thereof.

In one embodiment, the particle size of the active ingredient is less than or equal to about 60 microns, which can help to ensure uniformity of any blends of the particles with other ingredients, or to provide an adequate dispersion in a liquid vehicle.

The amount of drug absorbed depends on many factors. These factors include the drug concentration, the drug delivery vehicle, mucosal contact time, the venous drainage of the mucosal tissues, the degree that the drug is ionized at the pH of the absorption site, the size of the drug molecule, and its relative lipid solubility. Those of skill in the art can readily prepare an appropriate intranasal composition, which delivers an appropriate amount of the active agent, taking these factors into consideration.

### IV. Absorption Promoting Agents

The transport of the active ingredient across normal mucosal surfaces such as the nasal, buccal, or sublingual mucosa can be enhanced by optionally combining it with an absorption promoting agent, such as those disclosed in U.S. Pat. Nos. 5,629,011, 5,023,252, 6,200,591, 6,369,058, 6,380,175, and international Publication Number WO 01/60325, all of which are incorporated herein by reference with regard to absorption promoting agents. Examples of these absorption promoting agents include, but are not limited to, cationic polymers, surface active agents, chelating agents, mucolytic agents, cyclodextrin, polymeric hydrogels, combinations thereof, and any other similar absorption promoting agents known to those of skill in the art.

Representative absorption promoting excipients include phospholipids, such as phosphatidylglycerol or phosphatidylcholine, lysophosphatidyl derivatives, such as lysophosphatidylethanolamine, lysophosphatidylcholine, lysophosphatidylglycerol, lysophosphatidylserine, or lysophosphatidic acid, polyols, such as glycerol or propylene glycol, fatty acid esters thereof such as glycerides, amino acids, and esters thereof, and cyclodextrins. Gelling excipients or viscosity-increasing excipients can also be used.

### V. Mucoadhesive/Bioadhesive Polymers

The transport of the active ingredient across normal mucosal surfaces can also be enhanced by increasing the time in which the formulations adhere to the mucosal surfaces. Mucoadhesive/bioadhesive polymers, for example, those which form hydrogels, exhibit muco-adhesion and controlled drug release properties and can be included in the intranasal, buccal, and sublingual compositions described herein. Examples of such formulations are disclosed in U.S. Pat. Nos. 6,068,852 and 5,814,329; and International Publication Number WO99/58110, all of which are incorporated herein by reference with regard to such formulations.

Representative bioadhesive or hydrogel-forming polymers capable of binding to the nasal mucosa are well known to those of skill in the art, and include polycarbophil, polylysine, methylcellulose, sodium carboxymethylcellulose, hydroxypropyl-methylcellulose, hydroxyethyl cellulose, pectin, Carbopol 934P, polyethylene oxide 600K, Pluronic F127, polyisobutylene (PIB), polyisoprene (PIP), polyvinyl pyrrolidone (PVP), polyvinyl alcohol (PVA), xanthum gum, guar gum, and locust bean gum.

Other nasal delivery compositions are chitosan-based and are suitable to increase the residence time of the active ingredient on mucosal surfaces, which results in increasing its bioavailability. Examples of these nasal delivery compositions are disclosed in U.S. Pat. Nos. 6,465,626, 6,432,440, 6,391,318, and 5,840,341; European Patent Numbers EP0993483 and EP1051190; and International Publication Numbers WO 96/05810, WO 96/03142, and WO 93/15737, all of which are incorporated herein by reference with regard to nasal delivery compositions.

Additionally, the present invention can be formulated with powder microsphere and mucoadhesive compositions as disclosed in European Patent Numbers EP1025859 and EP1108423, which are incorporated herein by reference with regard to such composition. Finally, thiolated polymeric excipients that form covalent bonds with the cysteine-rich subdomains of the mucus membrane can also provide mucoadhesion, which prolongs the contact time between the active ingredient and the membrane. Such excipients are disclosed in International Publication Number WO 03/020771, which is incorporated herein by reference with regard to such excipients.

### VI. Preservatives

The intranasal compositions can also include one or more preservatives.

Representative preservatives include quaternary ammonium salts such as lauralkonium chloride, benzalkonium chloride, benzododecinium chloride, cetyl pyridium chloride, cetrimide, domiphen bromide; alcohols such as benzyl alcohol, chlorobutanol, o-cresol, phenyl ethyl alcohol; organic acids or salts thereof such as benzoic acid, sodium benzoate, potassium sorbate, parabens; or complex forming agents such as EDTA.

### VII. Other Excipients

The carriers and excipients include ion-exchange microspheres which carry suitable anionic groups such as carboxylic acid residues, carboxymethyl groups, sulphopropyl groups and methylsulphonate groups. Ion-exchange resins, such ascation exchangers, can also be used. Chitosan, which is partially deacetylated chitin, or poly-N-acetyl-D-glucosamine, or a pharmaceutically acceptable salt thereof such as hydrochloride, lactate, glutamate, maleate, acetate, formate, propionate, malate, malonate, adipate, or succinate.

Suitable other ingredients for use as non-ion-exchange microspheres include starch, gelatin, collagen and albumin.

The composition can also include an appropriate acid selected from the group consisting of hydrochloric acid, lactic acid, glutamic acid, maleic acid, acetic acid, formic acid, propionic acid, malic acid, malonic acid, adipic acid, and succinic acid. Other ingredients such as diluents are cellulose, microcrystalline cellulose, hydroxypropyl cellulose, starch, hydroxypropylmethyl cellulose, and the like.

Excipients to adjust the tonicity of the composition may be added such as sodium chloride, glucose, dextrose, mannitol, sorbitol, lactose, and the like.

Acidic or basic buffers can also be added to the intranasal composition to control the pH.

VIII. Incorporation of the Active Agent into the Compositions

In addition to using absorption enhancing agents, which increase the transport of the active agents through the mucosa, and bioadhesive materials, which prolong the contact time of the active agent along the mucosa, the administration of the active agent can be controlled by using controlled release formulations, which can provide rapid or sustained release, or both, depending on the formulations.

There are numerous particulate drug delivery vehicles known to those of skill in the art which can include the active ingredients, and deliver them in a controlled manner. Examples include particulate polymeric drug delivery vehicles, for example, biodegradable polymers, and particles formed of non-polymeric components. These particulate drug delivery vehicles can be in the form of powders, microparticles, nanoparticles, microcapsules, liposomes, and the like. Typically, if the active agent is in particulate form without added components, its release rate depends on the release of the active agent itself. Typically, the rate of absorption is enhanced by presenting the drug in a micronized form, wherein particles are below 20 microns in diameter. In contrast, if the active agent is in particulate form as a blend of the active agent and a polymer, the release of the active agent is controlled, at least in part, by the removal of the polymer, typically by dissolution, biodegradation, or diffusion from the polymer matrix.

The compositions can provide an initial rapid release of the active ingredient followed by a sustained release of the active ingredient. U.S. Pat. No. 5,629,011 provides examples of this type of formulation and is incorporated herein by reference with regard to such formulations. There are numerous compositions that utilize intranasal delivery and related methods thereof. Moreover, there are numerous methods and related delivery vehicles that provide for intranasal delivery of various pharmaceutical compositions. For example, intranasal compositions that employ current marketed nicotine replacement therapies (See, N. J. Benowitz, Drugs, 45: 157-170 (1993), which is incorporated herein by reference in its entirety) are also suitable for administering the metanicotines described herein. Nasal Insufflator Devices

### IX. Nasal Insufflator Devices

The intranasal compositions can be administered by any appropriate method according to their form. A composition including microspheres or a powder can be administered using a nasal insufflator device. Examples of these devices are well known to those of skill in the art, and include commercial powder systems such as Fisons Lomudal System. An insufflator produces a finely divided cloud of the dry powder or microspheres. The insufflator is preferably provided with a mechanism to ensure administration of a substantially fixed amount of the composition. The powder or microspheres can be used directly with an insulfator, which is provided with a bottle or container for the powder or microspheres. Alternatively, the powder or microspheres can be filled into a capsule such as a gelatin capsule, or other single dose device adapted for nasal administration. The insufflator preferably has a mechanism to break open the capsule or other device.

Further, the composition can provide an initial rapid release of the active ingredient followed by a sustained release of the active ingredient, for example, by providing more than one type of microsphere or powder.

### X. Use of Metered Sprays

Intranasal delivery can also be accomplished by including the active ingredient in a solution or dispersion in an aqueous medium which can be administered as a spray.

Appropriate devices for administering such a spray include metered dose aerosol valves and metered dose pumps, optionally using gas or liquid propellants.

Representative devices of this type are disclosed in the following patents, patent applications, and publications: WO 03/026559, WO 02/011800, WO 00/51672, WO 02/068029, WO 02/068030, WO 02/068031, WO 02/068032, WO 03/000310, WO 03/020350, WO 03/082393, WO 03/084591, WO 03/090812, WO 00/41755, and the pharmaceutical literature (See, Bell, A. Intranasal Delivery Devices, in Drug Delivery Devices Fundamentals and Applications, Tyle P. (ed), Dekker, New York, 1988), Remington's Pharmaceutical Sciences, Mack Publishing Co., 1975, all of which are incorporated herein by reference.

### XI. Other Modes of Intranasal Delivery

In addition to the foregoing, the compounds and intranasal compositions including the compounds can also be administered in the form of nose-drops, sprays, irrigations, and douches, as is known in the art.

Nose drops are typically administered by inserting drops while lying on a bed, with the patient on his or her back, especially with the head lying over the side of the bed. This approach helps the drops get farther back.

Nasal irrigation involves regularly flooding the nasal cavity with warm salty water, which includes one or more compounds as described herein, or their pharmaceutically acceptable salts.

Nasal douches are typically used by filling a nasal douche with a salt solution including one or more compounds as described herein, or their pharmaceutically acceptable salts, inserting the nozzle from the douche into one nostril, opening one's mouth to breathe, and causing the solution to flow into one nostril, rinse round the septum and turbinates, and discharge from the other nostril.

### XII. Buccal and Sublingual Compositions

Relative to an oral dosage form such as a tablet or capsule, buccal or sublingual delivery can also provide for rapid absorption, faster onset of therapeutic action and avoidance of liver or gut wall first pass metabolism. For patients who have difficulty in swallowing tablets, capsules or other solids or those who have intestinal failure, the buccal or sublingual delivery route is preferred.

Compositions for buccal administration include a metanicotine analog or pharmaceutically acceptable salt thereof and at least one excipient to form a solid dosage form with the metanicotine analog or pharmaceutically acceptable salt thereof. The solid dosage form disintegrates in an oral cavity with minimal liquid exposure and at body temperature, and ideally adheres to the body tissue of the oral cavity via direct adhesion to tissue or entrapment of the dosage form in-between the gum and inner cheek.

Compositions for sublingual administration include a metanicotine analog or pharmaceutically acceptable salt thereof and at least one excipient to form a solid dosage form. The solid dosage form disintegrates in an oral cavity at body temperature under the tongue.

The solid dosage forms can provide immediate release or controlled release or a combination thereof, wherein the dosage form disintegrates or melts in the oral cavity at body temperature with or without the aid of fluids, salivary fluids, mechanical erosion, or combinations thereof.

Alternatively, the dosage form can be sprayed into the oral cavity in the form of a solution spray or a dry powder.

Generally, the composition can be adhesive towards the body tissue lining the patient's oral cavity. The dosage form can be, but is not limited to, tablets, a bioadhesive patch or film, sponges, lozenges, hard candies, wafers, lollipops, sprays, gums, pills, pellets, spheres, combinations thereof, and other forms known to those of skill in the art.

There are numerous compositions and delivery vehicles suitable for buccal or sublingual delivery of the active ingredients. Examples of such compositions or delivery vehicles are disclosed in U.S. Pat. Nos. 6,676,959, 6,676,931, 6,593,317, 6,552,024, 6,306,914, 6,284,264, 6,248,358, 6,210,699, 6,177,096, 6,197,331, 6,153,222, 6,126,959, 6,286,698, 6,264,981, 6,187,323, 6,173,851, 6,110,486, 5,955,098, 5,869,082, 5,985,311, 5,948,430, 5,753,256, 5,487,902, 5,470,566, 5,362,489, 5,288,498, 5,288,497, 5,269,321, 6,488,953, 6,126,959, 6,641,838, 6,576,250, 6,509,036, 6,391,335, 6,365,182, 6,280,770, 6,221,392, 6,200,604, 6,531,112, and 6,485,706, all of which are incorporated herein by reference.

### XIII. Excipients for Buccal and Sublingual Compositions

In addition to the one or more active ingredients, other components of buccal and sublingual dosage forms include, but are not limited to, starch, mannitol, kaolin, calcium sulfate, inorganic salts, such as sodium chloride, powdered cellulose derivatives, dibasic and tribasic calcium phosphate, calcium sulfate, magnesium carbonate, magnesium oxide, poloxamers such as polyethylene oxide, hydroxypropyl methylcellulose, anionic excipients, cationic excipients, zwitterionic excipients, with reference to U.S. Pat. No. 6,436,950, which is incorporated herein by reference with regard to such excipients, polymeric hydrogel, powder microsphere mucoadhesive compositions, thiolated polymeric excipients, polycationic material, chitosan, cross-linked starches, fats, carbohydrates, polyols, buffers, phosphate buffers, acetate buffers, methocel, sodium chloride, water, lactic acid, benzalkonium chloride, demineralized water, cellulose, microcrystalline cellulose, hydroxypropyl cellulose, hydrogenated vegetable oil, flavoring agents, phospholipids, xylitol, cacao, combinations thereof, and other similar excipients known to those of skill in the art.

### XIV. Permeation Enhancers

Permeation enhancers can also be present. Representative permeation enhancers include, without limitation, 23-lauryl ether, aprontinin, azone, benzalkonium chloride, cetylpyridinium chloride, cetyltrimethylammonium bromide, cyclodextrin, dextran sulfate, lauric acid, lysophosphatidylcholine, menthol, sodium methoxysalicylate, methyloleate, oleic acid, phosphatidylcholine, polyoxyethylene, polysorbate, sodium EDTA, sodium glycocholate, sodium glycodeoxycholate, sodium lauryl sulfate, sodium salicylate, sodium taurocholate, sodium taurodeoxycholate, sulfoxides, short and medium chain mono-, di- and triglycerides and other polyol esters, and various alkyl glycosides.

### XV. Binders

Binders can also be present. Suitable binders include substances such as celluloses, including but not limited to cellulose, methylcellulose, ethylcellulose, hydroxypropyl cellulose and hydroxymethylcellulose, polypropylpyrrolidone, polyvinylpyrrolidone, gelatin, polyethylene glycol, starch, natural gums such as acacia, alginates, guar, and gum arabic) and synthetic gums and waxes.

### XVI. Lubricants

A lubricant is typically used in a tablet formulation to prevent the tablet and punches from sticking in the die. Suitable lubricants include calcium stearate, glyceryl monostearate, glyceryl behenate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc and zinc stearate. A preferred lubricant is magnesium stearate. The magnesium stearate is generally present in an amount from about 0.25 wt % to about 4.0% wt %.

### XVII. Disintegrants and Glidants

Other ingredients such as disintegrants and glidants can also be added to the composition to break up the dosage form and release the compound. Suitable disintegrants include sodium starch glycolate, sodium carboxymethyl cellulose, calcium carboxymethyl cellulose, croscarmellose sodium, polyvinylpyrrolidone, crospovidone, methyl cellulose, microcrystalline cellulose, powdered cellulose, lower alkyl-substituted hydroxypropyl cellulose, polacrilin potassium, starch, pregelatinized starch and sodium alginate. Of these, croscarmellose sodium and sodium starch glycolate are preferred, with croscarmellose sodium being most preferred. The croscarmellose sodium is generally present in an amount from about 0.5 wt % to about 6.0 wt %. The amount of disintegrant included in the dosage form will depend on several factors, including the properties of the dispersion, the properties of the porosigen, and the properties of the disintegrant selected. Generally, the disintegrant will comprise from 1 wt % to 15 wt %, preferably from 1 wt % to 10 wt % of the dosage form.

Suitable glidants include but are not limited to, silicon dioxide, talc, cornstarch, combinations thereof, and any other similar glidants known to those of skill in the art.

### XVIII. Methods of Treatment

The intranasal, buccal, or sublingual formulations can be used to treat or prevent a condition or disorder in a subject susceptible to such a condition or disorder. The method involves administering an effective amount of either (E)-metanicotine, (2S)-(4E)-N-methy-5-(3-(5-isopropoxypyridin)yl)-4-penten-2-amine, or (2S)-(4E)-N-methyl-5-(3-(5-methoxypyridin)yl)-4-penten-2-amine, or a pharmaceutically acceptable salt thereof.

The instant compounds are modulators of the α₄β₂ NNR subtype, characteristic of the CNS, and can be used for preventing or treating various conditions or disorders, including those of the CNS, in subjects which have or are susceptible to such conditions or disorders, by modulation of α₄β₂ NNRs. The compounds have the ability to selectively bind to the α₄β₂ NNRs and express nicotinic pharmacology, including the ability to act as partial agonists, agonists, antagonists, or inverse agonists. For example, compounds of the present invention, when administered in effective amounts to patients in need thereof, provide some degree of prevention of the progression of the CNS disorder, namely providing protective effects, amelioration of the symptoms of the CNS disorder, or amelioration of the reoccurrence of the CNS disorder.

The compounds can be used to treat or prevent those types of conditions and disorders for which other types of nicotinic compounds have been proposed as therapeutics. See, for example, Williams et al., Drug News Perspec. 7(4): 205 (1994), Americ et al., CNS Drug Rev. 1(1): 1-26 (1995), Americ et al., Exp. Opin. Invest. Drugs 5(1):79-100 (1996), Bencherif et al., J. Pharmacol. Exp. Ther. 279: 1413 (1996), Lippiello et al., J. Pharmacol. Exp. Ther. 279: 1422 (1996), Damaj et al., J. Pharmacol. Exp. Ther. 291: 390 (1999); Chiari et al., Anesthesiology 91: 1447 (1999), Lavand'homme and Eisenbach, Anesthesiology 91: 1455 (1999), Holladay et al., J. Med. Chem. 40(28): 4169-94 (1997), Bannon et al., Science 279: 77 (1998), PCT WO 94/08992, PCT WO 96/31475, PCT WO 96/40682, and U.S. Pat. Nos. 5,583,140 to Bencherif et al., 5,597,919 to Dull et al., 5,604,231 to Smith et al. and 5,852,041 to Cosford et al., the disclosures of which are incorporated herein by reference with regard to therapeutic pharmacology.

The compounds and their pharmaceutical compositions are useful in the treatment or prevention of a variety of CNS disorders, including neurodegenerative disorders, neuropsychiatric disorders, neurologic disorders, and addictions. The compounds and their pharmaceutical compositions can be used to treat or prevent attention disorders; to provide neuroprotection; to treat convulsions and multiple cerebral infarcts; to treat cognitive disorders, mood disorders, compulsions and addictive behaviors; to provide analgesia; to control inflammation, such as mediated by cytokines and nuclear factor kappa B, and treat inflammatory disorders; to provide pain relief, including, relief from acute pain, chronic pain, neurologic pain, neuropathic pain, female specific pain, post-surgical pain, or cancer pain; and to treat infections, such as anti-infectious agents for treating bacterial, fungal, and viral infections.

Exemplary disorders, diseases, and conditions that the compounds and pharmaceutical compositions of the present invention can be used to treat or prevent are: age-associated memory impairment, mild cognitive impairment, pre-senile dementia, also known as early onset Alzheimer's disease, senile dementia, also known as dementia of the Alzheimer's type, Lewy body dementia, HIV-dementia, vascular dementia, Alzheimer's disease, stroke, AIDS dementia complex, attention deficit disorder, attention deficit hyperactivity disorder, dyslexia, schizophrenia, schizophreniform disorder, schizoaffective disorder, cognitive deficits in schizophrenia, Parkinsonism including Parkinson's disease, Pick's disease, Huntington's chorea, tardive dyskinesia, hyperkinesia, progressive supranuclear palsy, Creutzfeld-Jakob disease, multiple sclerosis, amyotrophic lateral sclerosis, epilepsy, mania, anxiety, depression, panic disorders, bipolar disorders, generalized anxiety disorder, obsessive compulsive disorder, rage outbursts, Tourette's syndrome, autism, drug and alcohol addiction, tobacco addiction, obesity, cachexia, psoriasis, lupus, acute cholangitis, aphthous stomatitis, asthma, viral pneumonitis, arthritis including rheumatoid arthritis and osteoarthritis, endotoxaemia, sepsis, atherosclerosis, idiopathic pulmonary fibrosis, and neoplasias.

The present invention is believed useful in the treatment or prevention of diseases, disorders, and conditions, without appreciable adverse side effects, which side effects may include significant increases in blood pressure and heart rate, significant negative effects upon the gastrointestinal tract, and significant effects upon skeletal muscle. The compounds of the present invention, when employed in effective amounts, can modulate the activity of the α4β2 NNRs without appreciable interaction with the nicotinic subtypes that characterize the human ganglia, as demonstrated by their lack of ability to elicit nicotinic function in adrenal chromaffin tissue, or skeletal muscle, as demonstrated by their lack of ability to elicit nicotinic function in cell preparations expressing muscle-type nicotinic receptors. Thus, these compounds are capable of treating or preventing diseases, disorders, and conditions without eliciting significant side effects associated with activity at ganglionic and neuromuscular sites. Thus, administration of the compounds provides a therapeutic window in which treatment or prevention of certain diseases, disorders, and conditions is provided, and certain side effects are avoided. That is, an effective dose of the compound is sufficient to provide the desired effects upon the disease, disorder, or condition, but is insufficient, namely is not at a high enough level, to provide undesirable side effects.

The following examples are provided to illustrate the present invention, and should not be construed as limiting. In these examples, all parts and percentages are by weight, unless otherwise noted.

### EXAMPLES

### Example 1

### Measurement of Drug Absorption

The “buccal absorption test,” as is known in the art, can be used to measure the kinetics of drug absorption. The methodology involves the swirling of a 2.5 mL sample of the test solution for up to 15 minutes by human volunteers, followed by the expulsion of the solution. The amount of drug remaining in the expelled volume is then determined in order to assess the amount of drug absorbed. The appreciated drawbacks of this method include salivary dilution of the drug, accidental swallowing of a portion of the sample solution, and the inability to localize the drug solution within a specific site (buccal, sublingual, or gingival) of the oral cavity. Various modifications of the buccal absorption test have been carried out correcting the salivary dilution and accidental swallowing, but these modifications also suffer from the inability of site localization. A feasible approach to achieve absorption site localization is to retain the drug on the buccal mucosa using a bioadhesive system. Pharmacokinetic parameters such as bioavailability can then be calculated from the plasma concentration vs. time profile.

Another in vivo method includes that carried out using a small perfusion chamber attached to the upper lip of anesthetized dogs. The perfusion chamber is attached to the tissue by cyanoacrylate cement. The drug solution is circulated through the device for a predetermined period of time and sample fractions are then collected from the perfusion chamber in order to determine the amount of drug remaining in the chamber, and blood samples are drawn after 0 and 30 minutes in order to determine amount of drug absorbed across the mucosa.

Each of these methods is described for reference and, prophetically, the metanicotine analogs herein described are believed to demonstrate absorption in such methods.

### Example 2

### Ability of Test Compounds A and B to Permeate Cultured Human Respiratory Tissue In Vitro

The objective of this study was to determine the apical-to-basolateral permeability of two test compounds ((2S)-(4E)-N-methyl-5-(3-(5-methoxypyridin)yl)-4-penten-2-amine, (“Compound A”), and N-methyl-(4-pyridin-3-yl-but-3-enyl)-amine ((E)-metanicotine), (“Compound B”)) across human respiratory tissue culture in vitro in the absence and presence of enzymatic inhibitors.

### Study Design and Methodology

### Materials

Compounds A and B, were supplied by Targacept, Inc. (Winston Salem, N.C.), as their hemigalactarate salts. Atenolol, antipyrine, pargyline (MAO inhibitor), quinidine (CYP2D6 inhibitor), phenylethylamine (PEA), and Bufuralol were obtained from Sigma-Aldrich (St. Louis, Mo.). The nasal tissue culture (EpiAir®) and Dulbeccos' Phosphate Buffered Saline (OPBS) were obtained from MatTek Corporation (Ashland, Mass.).

### Permeation Across Respiratory Epithelium In Vitro

An in vitro model of MatTek EpiAir® tissue was used for the permeability assessment of the test articles and control compounds. The EpiAir® culture consists of cells that have been cultured to form a pseudo-stratified, highly differentiated model closely resembling the epithelial tissue of the human respiratory tract. The histological cross-sections of the cultured tissue reveal a pseudo-stratified mucociliary phenotype.

### Procedure

EpiAir® tissues plated in 12 well plates were pre-equilibrated for the assay by culturing them for 24 hours at 37° C. with 5% CO2 in a humidified incubator. On the day of the assay, the cultures were washed two times with Dulbeccos' Phosphate Buffered Saline (DPBS buffer) at pH 7.4, and then dosed with donor and receiver buffer applied to the apical and basolateral surfaces of the culture, respectively. The receiver buffer consisted of DPBS, at pH 7.4. The donor solution consisted of DPBS buffer containing the appropriate compounds as specified in Table 1. All treatments were performed in triplicate. The receiver buffer was sampled at 15, 30, 60, and 120 minutes, and the donor buffer was sampled at 120 minutes.

TABLE 1
Summary of the Study Treatment Groups and Dosed Compounds
Example 2

| | Target Concentrations | Average Measured Concentrations |
|---|---|---|
| Treatment Group | in Dosing Solution | in Dosing Solution |
| 1. Compound B + | Compound B: 100 μM | Compound B: 102.7 μM |
| Controls: Atenolol, Caffeine, | Atenolol: 100 μM | Atenolol: 101.1 μM |
| and Lucifer Yellow | Caffeine: 100 μM | Caffeine: 93.7 μM |
| | Lucifer Yellow: 100 μM | Lucifer Yellow: 105.4 μM |
| 2. Compound B + | Compound B: 100 μM | Compound B: 97.6 μM |
| Controls: Atenolol, Caffeine, | Atenolol: 100 μM | Atenolol: 97.9 μM |
| and Lucifer Yellow + | Caffeine: 100 μM | Caffeine: 97.2 μM |
| MAO B Inhibitor: Parglyine | Lucifer Yellow: 100 μM | Lucifer Yellow: 106.1 μM |
| | Pargyline: 50 μM | |
| 3. Phenylethylamine (PEA) + | PEA: 100 μM | PEA: 106.7 μM |
| Controls: Atenolol, Caffeine, | Atenolol: 100 μM | Atenolol: 98.0 μM |
| and Lucifer Yellow | Caffeine: 100 μM | Caffeine: 98.3 μM |
| | Lucifer Yellow: 100 μM | Lucifer Yellow: 108.7 μM |
| 4. Phenylethylamine (PEA) + | PEA: 100 μM | PEA: 103.3 μM |
| Controls: Atenolol, Caffeine, | Atenolol: 100 μM | Atenolol: 94.9 μM |
| and Lucifer Yellow + | Caffeine: 100 μM | Caffeine: 100.5 μM |
| MAO B Inhibitor: Pargyline | Lucifer Yellow: 100 μM | Lucifer Yellow: 123.7 μM |
| | Pargyline: 50 μM | |
| 5. Compound A + | Compound A: 100 μM | Compound A: 98.0 μM |
| Controls: Atenolol, Caffeine, | Atenolol: 100 μM | Atenolol: 106.3 μM |
| and Lucifer Yellow | Caffeine: 100 μM | Caffeine: 101.1 μM |
| | Lucifer Yellow: 100 μM | Lucifer Yellow: 103.9 μM |
| 6. Compound A + | Compound A: 100 μM | Compound A: 97.3 μM |
| Controls: Atenolol, Caffeine, | Atenolol: 100 μM | Atenolol: 101.6 μM |
| and Lucifer Yellow + | Caffeine: 100 μM | Caffeine: 103.3 μM |
| CYP2D6 Inhibitor: Quinidine | Lucifer Yellow: 100 μM | Lucifer Yellow: 108.3 μM |
| | Quinidine: 50 μM | |
| 7. Bufuralol + | Bufuralol: 100 μM | Bufuralol: 98.9 μM |
| Controls: Atenolol, Caffeine, | Atenolol: 100 μM | Atenolol: 105.7 μM |
| and Lucifer Yellow | Caffeine: 100 μM | Caffeine: 98.5 μM |
| | Lucifer Yellow: 100 μM | Lucifer Yellow: 109.0 μM |
| 8. Bufuralol + | Bufuralol: 100 μM | Bufuralol: 102.3 μM |
| Controls: Atenolol, Caffeine, | Atenolol: 100 μM | Atenolol: 101.0 μM |
| and Lucifer Yellow + | Caffeine: 100 μM | Caffeine: 104.0 μM |
| CYP2D6 Inhibitor: Quinidine | Lucifer Yellow: 100 μM | Lucifer Yellow: 106.7 μM |
| | Quinidine: 50 μM | |

### Sample and Data Analysis

The Lucifer Yellow concentration in the receiver samples was measured using a FluoStar fluorescence plate reader (BMG Laboratories, Durham, N.C.). The excitation and emission wavelengths were 485 and 538 nm, respectively. Test articles, atenolol, caffeine, PEA, and Bufuralol were analyzed by LC/MS/MS.

The apparent permeability coefficient, Pₐₚₚ, was calculated as follows:

Pₐₚₚ=(dCᵣ/dt)×Vᵣ/(A×Cₒ)

where:
dCᵣ/dt is the slope of the cumulative concentration in the receiver compartment versus time;
Vᵣ is the volume of the receiver compartment;
A is the surface area of epithelium available for permeation; and
Cₒ is the dosing solution concentration.

### Results

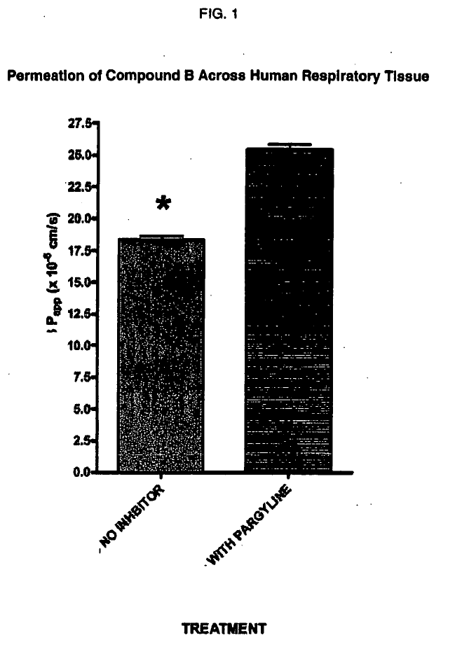

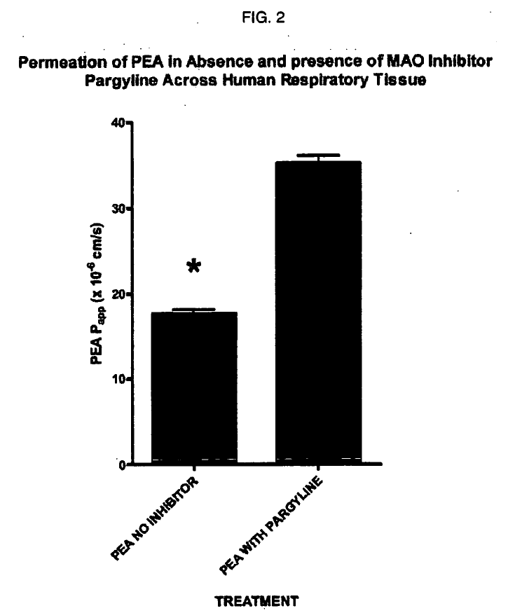

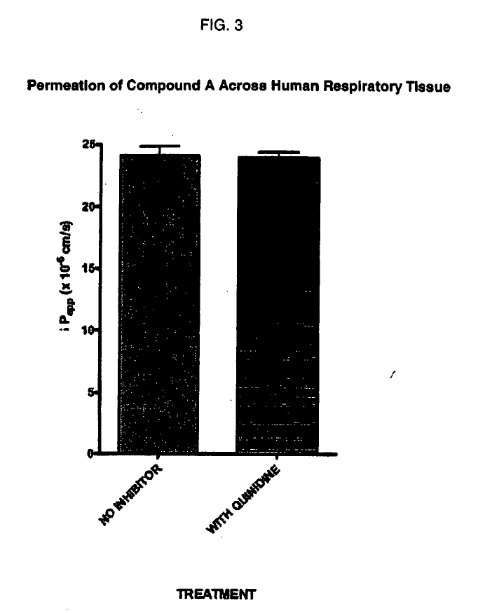

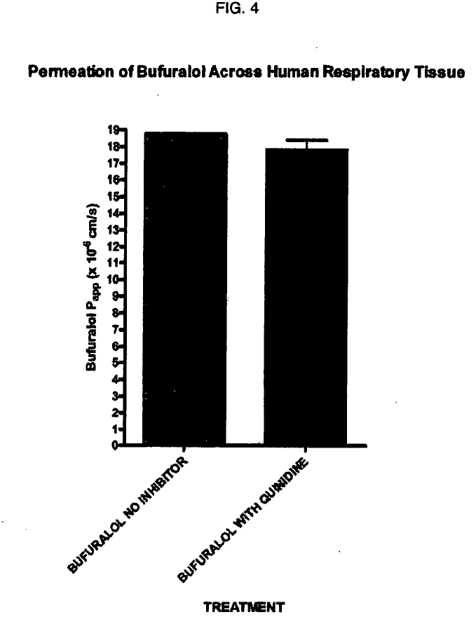

Individual results for each treatment are in Table 2. FIGS. 1 and 2 summarize permeability results for Compound B and MAO substrate Phenylethylamine, respectively. FIGS. 3 and 4 summarize permeability results for Compound A and CYP2D6 substrate Bufuralol, respectively.

TABLE 2
Assay Results

| | Rep 1 | Rep 2 | Rep 3 | Average ± STD |
|---|---|---|---|---|
| TREATMENT 1 | | | | |
| Compound B Assayed With Controls | | | | |
| Lucifer Yellow Pₐₚₚ (×10⁻⁶ cm/s) | 14.81 | 14.11 | 14.26 | 14.40 ± 0.37 |
| Atenolol Pₐₚₚ (×10⁻⁶ cm/s) | 17.42 | 16.80 | 15.88 | 16.70 ± 0.78 |
| Caffeine Pₐₚₚ (×10⁻⁶ cm/s) | 36.65 | 37.29 | 38.07 | 37.34 ± 0.71 |
| Compound B Pₐₚₚ (×10⁻⁶ cm/s) | 18.51 | 17.98 | 18.42 | 18.31 ± 0.28 |
| TREATMENT 2 | | | | |
| Compound B Assayed With Controls + | | | | |
| MAO Inhibitor Pargyline | | | | |
| Lucifer Yellow Pₐₚₚ (×10⁻⁶ cm/s) | 15.09 | 15.00 | 15.21 | 15.10 ± 0.11 |
| Atenolol Pₐₚₚ (×10⁻⁶ cm/s) | 16.71 | 16.85 | 16.59 | 16.72 ± 0.13 |
| Caffeine Pₐₚₚ (×10⁻⁶ cm/s) | 36.16 | 39.60 | 39.75 | 38.50 ± 2.03 |
| Compound B Pₐₚₚ (×10⁻⁶ cm/s) | 25.12 | 25.87 | 25.19 | 25.39 ± 0.41 |
| TREATMENT 3 | | | | |
| MAO Substrate Phenylethylamine | | | | |
| Assayed With Controls | | | | |
| Lucifer Yellow Pₐₚₚ (×10⁻⁶ cm/s) | 15.61 | 13.88 | 14.99 | 14.82 ± 0.88 |
| Atenolol Pₐₚₚ (×10⁻⁶ cm/s) | 17.22 | 16.88 | 16.80 | 16.97 ± 0.23 |
| Caffeine Pₐₚₚ (×10⁻⁶ cm/s) | 36.80 | 34.85 | 37.32 | 36.32 ± 1.30 |
| Phenylethylamine Pₐₚₚ (×10⁻⁶ cm/s) | 18.84 | 18.94 | 19.04 | 18.94 ± 0.10 |
| TREATMENT 4 | | | | |
| MAO Substrate Phenylethylamine | | | | |
| Assayed With Controls + MAO Inhibitor | | | | |
| Pargyline | | | | |
| Lucifer Yellow Pₐₚₚ (×10⁻⁶ cm/s) | 13.270 | 13.82 | 14.20 | 13.76 ± 0.46 |
| Atenolol Pₐₚₚ (×10⁻⁶ cm/s) | 18.13 | 19.02 | 19.19 | 18.78 ± 0.57 |
| Caffeine Pₐₚₚ (×10⁻⁶ cm/s) | 41.10 | 37.31 | 41.08 | 39.83 ± 2.18 |
| Phenylethylamine Pₐₚₚ (×10⁻⁶ cm/s) | 34.52 | 36.30 | 34.72 | 35.18 ± 0.98 |
| TREATMENT 5 | | | | |
| Compound A Assayed With Controls | | | | |
| Lucifer Yellow Pₐₚₚ (×10⁻⁶ cm/s) | 14.91 | 13.09 | 15.68 | 14.56 ± 1.33 |
| Atenolol Pₐₚₚ (×10⁻⁶ cm/s) | 16.62 | 15.95 | 16.67 | 16.41 ± 0.40 |
| Caffeine Pₐₚₚ (×10⁻⁶ cm/s) | 42.00 | 37.99 | 38.34 | 39.45 ± 2.22 |
| Compound A Pₐₚₚ (×10⁻⁶ cm/s) | 25.13 | 22.50 | 24.40 | 24.01 ± 1.36 |
| TREATMENT 6 | | | | |
| Compound A Assayed With Controls + | | | | |
| CYP2D6 Inhibitor Quinidine | | | | |
| Lucifer Yellow Pₐₚₚ (×10⁻⁶ cm/s) | 13.52 | 14.54 | 15.29 | 14.45 ± 0.89 |
| Atenolol Pₐₚₚ (×10⁻⁶ cm/s) | 17.41 | 17.00 | 17.02 | 17.15 ± 0.23 |
| Caffeine Pₐₚₚ (×10⁻⁶ cm/s) | 34.79 | 36.78 | 36.41 | 36.00 ± 1.06 |
| Compound A Pₐₚₚ (×10⁻⁶ cm/s) | 22.98 | 24.26 | 24.43 | 23.89 ± 0.79 |
| TREATMENT 7 | | | | |
| CYP2D6 Substrate Bufuralol Assayed | | | | |
| With Controls | | | | |
| Lucifer Yellow Pₐₚₚ (×10⁻⁶ cm/s) | 13.55 | 13.57 | 14.52 | 13.88 ± 0.56 |
| Atenolol Pₐₚₚ (×10⁻⁶ cm/s) | 16.76 | 16.72 | 17.12 | 16.87 ± 0.22 |
| Caffeine Pₐₚₚ (×10⁻⁶ cm/s) | 41.77 | 37.58 | 43.44 | 40.93 ± 3.02 |
| Bufuralol Pₐₚₚ (×10⁻⁶ cm/s) | 18.66 | 18.87 | 18.69 | 18.74 ± 0.11 |
| TREATMENT 8 | | | | |
| CYP2D6 Substrate Bufuralol Assayed | | | | |
| With Controls + CYP2D6 Inhibitor | | | | |
| Quinidine | | | | |
| Lucifer Yellow Pₐₚₚ (×10⁻⁶ cm/s) | 13.45 | 13.85 | 14.69 | 14.00 ± 0.63 |
| Atenolol Pₐₚₚ (×10⁻⁶ cm/s) | 17.94 | 17.09 | 17.38 | 17.47 ± 0.43 |
| Caffeine Pₐₚₚ (×10⁻⁶ cm/s) | 37.66 | 38.24 | 38.90 | 38.27 ± 0.62 |
| Bufuralol Pₐₚₚ (×10⁻⁶ cm/s) | 18.43 | 18.34 | 16.67 | 17.81 ± 0.99 |

There appears to be a good correlation between Pₐₚₚ values across the EpiAir® model and in vivo AUC values following intranasal application (see, Leonard et al., 2005, herein incorporated by reference with regard to such study). Results of the current study demonstrate that in all tissue culture replicates, the Pₐₚₚ value of atenolol, which is a moderately absorbed compound, was approximately one half that of caffeine, which is a highly absorbed compound. In addition, across all treatments, the average atenolol and caffeine Pₐₚₚ values were similar, as well as the Pₐₚₚ values of the culture integrity marker Lucifer Yellow that attests to the functionality of the model and good reproducibility of permeability results.

Compound B permeation in the absence or in the presence of a MAO B inhibitor, pargyline, was less than the permeation of caffeine, but higher than the permeation of atenolol (see Table 2). Therefore, regardless of enzymatic inhibition, Compound B can be expected to have a medium-to-high absorption across nasal tissue. Test article Compound B had a significantly lower Pₐₚₚ value in the absence, than in the presence, of the MAO B inhibitor pargyline (see Table 2, FIG. 1). In addition, similar results were obtained for a MAO B substrate, phenylethylamine (“PEA”), which confirms the presence of a MAO B-mediated metabolism in the tissue (see Table 2 and FIG. 2). These results suggest that the MAO B-mediated Compound B metabolism can limit Compound B permeation across human respiratory mucosa and, therefore, may affect Compound B delivery in vivo.

The permeation of Compound A across the tissue culture in the absence or presence of CYP2D6 inhibition was lower than the permeation of caffeine, but higher than the permeation of atenolol (see Table 2). Therefore, Compound A can have a medium-to-high absorption across nasal tissue in vivo. Administration of a CYP2D6 inhibitor, quinidine, did not affect the permeation of Compound A, or the permeation of a CYP2D6 marker substrate, bufuralol (see Table 2, FIGS. 3 and 4). Therefore, CYP2D6-mediated metabolism appears not to be a limiting factor for drug permeation across a human respiratory tissue culture. These results are in accordance with the published lack of CYP2D6 mRNA expression in human respiratory mucosa (see, Mace et al., 1998, herein incorporated by reference with regard to such study).

Intranasal delivery has utility for administration of central nervous system (CNS) drugs such as opioids (see, Rudy et al, 2004, herein incorporated by reference with regard to such teaching) and antimuscarinic agents (see, Ahmed et al., 2000, herein incorporated by reference with regard to such teaching). Therefore, drugs that have a substantial potential to cross the nasal mucosa, as well as the blood-brain barrier (“BBB”), may have a good CNS delivery profile. Compound B exhibited a moderate-to-high potential to cross the respiratory epithelium, which was limited by MAO activity, and may have an improved CNS delivery in the presence of an MAO inhibitor. Compound A exhibited a moderate-to-high permeation across the respiratory epithelium, which was independent from the CYP2D6 metabolism. Therefore, CNS penetration of Compound A following intranasal application may not be enhanced by co-administration of the CYP2D6 inhibitor.

Further reference is made to Ahmed, S., Sileno, A. P., deMeireles, J. C., Dua, R., Pimplaskar, H. K., Xia, W. J., Marinaro, J., Langrback, E., Matos, F. J., Putcha L., Romeo, V. D., and Behl, C. R. (2000) Pharm. Res. 17: 974-977; Leonard, A. K., Sileno, A. P., Macevilly, C., Foerder, C. A., Quay, S. C., and Costantino, H. R. (2005) J. Pharm. Sci. 94: 1736-1746; Mace, K., Bowman, E. D., Vautravers, P., Shields, P. G., Harris, C. C., and Pfeifer, A. M. (1998) Eur. J. Cancer 34: 914-920; and Rudy, A. C., Coda, B. A., Archer, S. M., and Wermeling D. P. (2004) Anesth. Analg. 99: 1379-1386, each of which is herein incorporated by reference with regard to such teachings.

### Example 3

### Brain Penetration of Compounds A and B

### Study Objective

The objective of this study was to determine the brain penetration potential of Compounds A and B using in situ brain perfusion in the absence and presence of enzymatic inhibitors.

### Study Design and Methodology

### Materials

Compounds A and B were supplied by Targacept, Inc. (Winston Salem, N.C.). Atenolol, antipyrine, pargyline (MAO inhibitor), quinidine (CYP2D6 inhibitor), and Kreb's Ringer Bicarbonate buffer (KRB) were obtained from Sigma-Aldrich (St. Louis, Mo.).

### Animals

Animals used in this study were Sprague-Dawley rats (approximate weight 250-300 grams), obtained from Hilltop Lab Animals, Scottdale, Pa. Upon arrival, the rats were assigned randomly to treatment groups and acclimated for at least 24 hours. The animals were housed two per cage and identified by cage labels. A single room was used for this study. The animals were supplied with water and a commercial rodent diet ad libitum. On the day of the experiment, each rat was anesthetized intraperitoneally with Ketamine HCl/Xylazine HCl solution prior to being implanted with a cannula into the left carotid artery. Branch arteries were bed, and the cardiac supply was cut off prior to brain perfusion.

### Brain Perfusion

Perfusion was performed using the single time-point method. The perfusate, composed of KRB buffer containing the two control compounds, atenolol and antipyrine, and one test article in the absence or presence of the appropriate inhibitor, was infused into the animals via the left external carotid artery for 30 seconds by an infusion pump. Following 30 seconds of perfusion, the pump was stopped, and the brain was removed from the skull immediately. The brain was cut longitudinally in hag. Each left cerebral hemisphere was placed into a chilled tube, frozen on dry ice, and stored frozen at −60° C. to −80° C. until analyzed. Four rats were perfused to allow for exclusion of data from one rat if the control, atenolol, clearly indicated experimental failure. Thus, data from the first three successful experiments, according to sequential rat number, are reported. Atenolol was perfused at a 50 μM concentration, and antipyrine was perfused at a 5 μM concentration. Test articles were perfused at concentrations of 50 μM. The outline of the experimental treatments and the target and measured concentrations of the test articles and control compounds in the perfusate are presented in Table 3.

TABLE 3
Summary of the Study Treatment Groups and Concentrations Perfused

| | Target Perfusate | Measured Perfusate |
|---|---|---|
| Treatment Group | Concentrations (μM) | Concentrations (μM) |
| 1. Compound B + | Compound B: 50 | Compound B: 60.9 |
| Controls, Atenolol | Atenolol: 50 | Atenolol: 55.7 |
| and Antipyrine | Antipyrine: 5 | Antipyrine: 6.3 |
| 2. Compound B + | Compound B: 50 | Compound B: 61.1 |
| Controls, Atenolol | Atenolol: 50 | Atenolol: 52.6 |
| and Antipyrine + | Antipyrine: 5 | Antipyrine: 5.9 |
| Pargyline* | | |
| 1. Compound B + | Compound B: 50 | Compound B: 55.6 |
| Controls, Atenolol | Atenolol: 50 | Atenolol: 53.1 |
| and Antipyrine | Antipyrine: 5 | Antipyrine: 6.2 |
| 2. Compound B + | Compound B: 50 | Compound B: 54.9 |
| Controls, Atenolol | Atenolol: 50 | Atenolol: 53.0 |
| and Antipyrine + | Antipyrine: 5 | Antipyrine: 5.8 |
| Quinidine* | | |

*Target concentration of inhibitors pargyline and quinidine was 50 μM.

### Analysis of Samples and Data

The left brain hemisphere from each rat was thawed and weighed. Methanol (20% aqueous) was added to each left brain hemisphere at approximately 4 mL per 1 g of brain tissue, and the mixture was homogenized using sonication with a VirSonic Ultrasonic Cell Disruptor 100 (VirTis). The test articles and two reference compounds were analyzed in the resulting homogenate by using LC/MS/MS.

The unidirectional brain transfer constants Kᵢₙ (mL/g/min) were determined for the test articles and the high permeability reference, antipyrine, using the following equation for the single-point perfusion assay:

Kᵢₙ=[C_{br}/C_{pf}]/t

where:
C_{br}/C_{pf} is the apparent brain distribution volume (mL/g of brain tissue);
C_{br} is the concentration of drug in the brain tissue (pmol of drug per g of brain tissue);
C_{pf} is the drug concentration in the perfusion fluid (pmol/mL of perfusate); and
t is the net perfusion time (minutes).

To exclude the drug contained in the capillary space from the brain concentration values, the apparent brain distribution volume of atenolol was subtracted from the drug values in each animal. Graphic representation and statistical analyses of the data were performed using GraphPad Prism software.

### Results

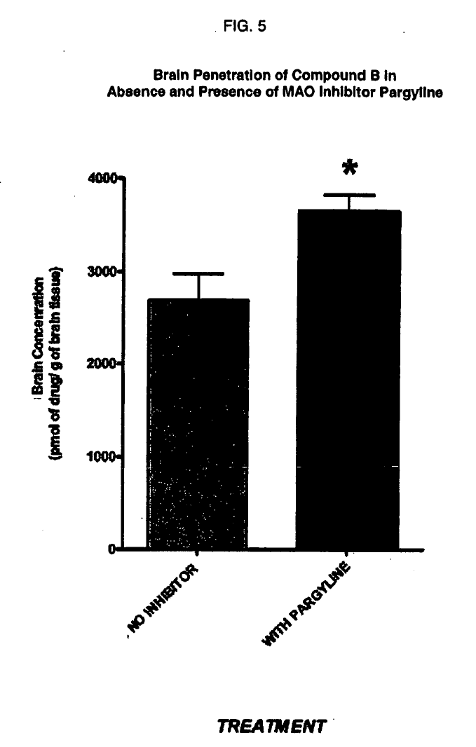

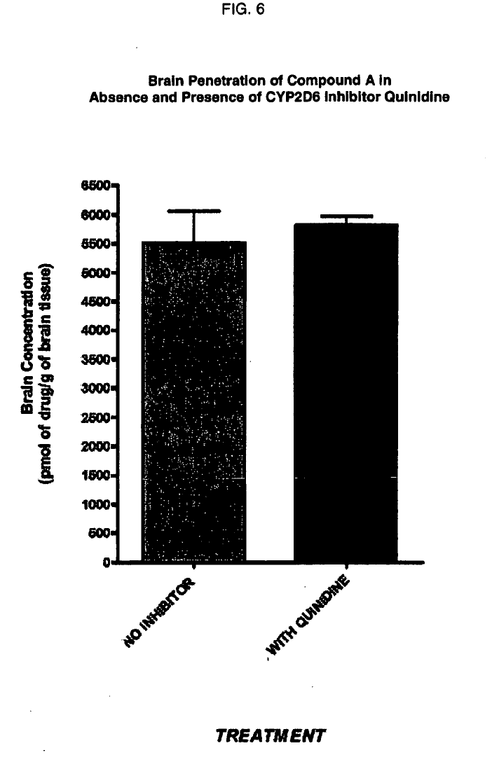

Individual results for each treatment representing brain concentrations were corrected for vascular content as well as Kᵢₙ values and are in Tables 4 and 5. FIGS. 5 and 6 summarize perfusion results for Compound B and Compound A, respectively. As shown in FIG. 5, in the presence of the MAO inhibitor, pargyline, the brain concentration of Compound B was significantly increased compared to the Compound B brain concentration in the absence of pargyline (*p<0.05, two-tailed t-test). As shown in FIG. 6, there was no significant difference between Compound A brain concentrations when perfused in the absence or in the presence of the CYP2D6 inhibitor, quinidine.

TABLE 4
Compound B Assay Results

| | Rat 1 | Rat 2 | Rat 3 | Average ± STD |
|---|---|---|---|---|
| TREATMENT 1 | | | | |
| Compound B Perfused with References | | | | |
| Atenolol Vascular Space (μL/g) | 15.80 | 11.66 | 13.69 | 13.72 ± 2.07 |
| Antipyrine Brain Concentration* (pmol/g) | 1123.1 | 1200.2 | 926.0 | 1083.1 ± 141.4 |
| Antipyrine Kᵢₙ (mL/g/min) | 0.355 | 0.379 | 0.292 | 0.342 ± 0.045 |
| Compound B Brain Concentration* (pmol/g) | 2725.9 | 3170.1 | 2147.0 | 2681.0 ± 513.0 |
| Compound B Kᵢₙ (mL/g/min) | 0.090 | 0.104 | 0.071 | 0.088 ± 0.017 |
| TREATMENT 2 | | | | |
| Compound B Perfused with References + | | | | |
| MAO Inhibitor Pargyline | | | | |
| Atenolol Vascular Space (μL/g) | 16.59 | 13.69 | 10.89 | 13.72 ± 2.85 |
| Antipyrine Brain Concentration* (pmol/g) | 833.1 | 914.3 | 918.0 | 888.5 ± 47.9 |
| Antipyrine Kᵢₙ (mL/g/min) | 0.282 | 0.309 | 0.310 | 0.300 ± 0.016 |
| Compound B Brain Concentration* (pmol/g) | 3358.2 | 3655.8 | 3944.4 | 3652.8\hat{ } ± 293.1 |
| Compound B Kᵢₙ (mL/g/min) | 0.110 | 0.120 | 0.129 | 0.120\hat{ } ± 0.010 |

*Corrected for drug retained in vascular volume that did not enter the brain tissue.
\hat{ }Significantly higher than the corresponding values of Compound B in the absence of enzymatic inhibitor (p < 0.05, two-tailed t-test).

TABLE 5
Compound A Assay Results

| | Rat 1 | Rat 2 | Rat 3 | Average ± STD |
|---|---|---|---|---|
| TREATMENT 1 | | | | |
| Compound A Perfused with References | | | | |
| Atenolol Vascular Space (μL/g) | 12.84 | 10.70 | 12.96 | 12.17 ± 1.27 |
| Antipyrine Brain Concentration* (pmol/g) | 1426.6 | 1074.9 | 969.7 | 1157.1 ± 239.3 |
| Antipyrine Kᵢₙ (mL/g/min) | 0.463 | 0.349 | 0.315 | 0.376 ± 0.078 |
| Compound A Brain Concentration* (pmol/g) | 6558.8 | 5279.1 | 4675.5 | 5504.5 ± 961.7 |
| Compound A Kᵢₙ (mL/g/min) | 0.236 | 0.190 | 0.168 | 0.198+ ± 0.035 |
| TREATMENT 2 | | | | |
| Compound A Perfused with References + | | | | |
| CYP2D6 Inhibitor Quinidine | | | | |
| Atenolol Vascular Space (μL/g) | 13.18 | 17.93 | 15.23 | 15.45 ± 2.38 |
| Antipyrine Brain Concentration* (pmol/g) | 977.7 | 1244.5 | 1151.8 | 1124.7 ± 135.5 |
| Antipyrine Kᵢₙ (mL/g/min) | 0.336 | 0.427 | 0.395 | 0.386 ± 0.047 |
| Compound A Brain Concentration* (pmol/g) | 5574.4 | 6113.5 | 5721.1 | 5803.0 ± 278.8 |
| Compound A Kᵢₙ (mL/g/min) | 0.203 | 0.223 | 0.209 | 0.212 ± 0.010 |

*Corrected for drug retained in vascular volume that did not enter the brain tissue.
+No significant differences between the Kin values for Compound A in the absence and in
the presence of the inhibitor.

### Analytical Methodology

### Blank Brain Homogenate

Blank brain homogenate was prepared for use as a diluent for the standard curve and QC preparation. Two whole rat brains were placed in a 50 mL centrifuge tube. To this, 16 mL of 20:80 (v/v) methanol/water was added. The brains were then homogenized using a VirSonic 100 Ultrasonic Cell Disruptor. This procedure was repeated until sufficient homogenate was produced. The products of each homogenization were combined in 50 mL centrifuge tubes and frozen at −80° C. until needed for the analysis.

### Homogenization of Rat Left Brain Study Samples

Left brain samples were thawed, and their weights were recorded. To each sample, 4 mL of 20:80 (v/v) methanol/water was added. Each sample was then homogenized using a VirSonic 100 Ultrasonic Cell Disruptor. After homogenization the volume of each sample was recorded, and the samples were frozen at −80° C. until needed for analysis.

### Pre-Study Validation for Compounds A and B

To determine the accuracy and precision for the extraction of Compounds A and B from the rat brain homogenate, the analytical method was subjected to a one-day pre-study validation. A single standard curve and six replicates each of the three quality control levels (18 total) were extracted using the methodology outlined below and analyzed prior to study sample analysis.

### Standards and Quality Control Sample Preparation

To determine the concentration of Compound A, Compound B, atenolol, and antipyrine in brain homogenate samples, the standards were prepared in blank rat brain homogenate. Plastic tubes were used for all steps. Standards were prepared at concentrations of 1.0, 0.5, 0.25, 0.10, 0.050, 0.025, 0.010, or 0.005 μM by serial dilution. Quality control samples were also prepared at 0.50, 0.10, and 0.010 μM. Compound B was analyzed individually, while Compound A, atenolol, and antipyrine were pooled together for simultaneous analysis. Brain standards and quality control samples were treated identically to the brain samples.

### Sample Preparation for Brain Homogenate Samples

### Compound B

Extraction of brain standards, quality control samples, and study samples for Compound B was carried out on a Tomtec Quadra 96-Model 320 liquid handling system in a 96-well plate format. Brain samples (200 μL) were loaded onto a 96 well plate and then transferred to 400 μL of pure acetonitrile in a Sirocco Protein Precipitation Plate (Waters Corporation). The resulting suspensions were mixed and then filtered into a clean 96-well collection plate using a vacuum. A 200 μL aliquot of the resulting filtrate was then transferred to plastic HPLC vials for analysis.

### Compound A, Atenolol, and Antipyrine

Extraction of brain standards, quality control samples, and study samples for Compound A, atenolol, and antipyrine was carried out on a Tomtec Quadra 96-Model 320 liquid handling system in a 96-well plate format. Brain samples (200 μL) were loaded onto a 96 well plate and then transferred to 400 μL of acetonitrile containing internal standard (100 ng/mL Pindolol) in a Sirocco Protein Precipitation Plate (Waters Corporation). The resulting suspensions were mixed and then filtered into a clean 96-well collection plate using a vacuum. The resulting filtrates were then evaporated to dryness under nitrogen at 37° C. The resulting residues were reconstituted with 200 μL of water. The samples were then mixed, centrifuged, and transferred (100 μL) to plastic HPLC vials for analysis.

In all animals, the vascular space marked by atenolol, a compound that does not penetrate the brain, did not exceed 20 μL/g of brain tissue. These results indicate preserved blood-brain barrier properties during perfusion. In addition, the average Kᵢₙ values of anypyrine, a drug with high brain penetration potential, were consistent in all experimental groups ranging from 0.30 to 0.39 mL/g of brain tissue/min. These Kᵢₙ antipyrine values are similar to those reported and typically obtained using the brain perfusion technique (see Youdim et al., Flavanoid permeability across an in situ model of the blood-brain barrier, Free radic Biol med, 36: 592-604, 2004, herein incorporated by reference with regard to such teaching).

Both test compounds had lower Kᵢₙ values than co-perfused antipyrine, both in the absence and in the presence of enzymatic inhibitors. See FIGS. 5 and 6. Kᵢₙ values of Compound A and B are more than 100 times higher than those reported for drugs that do not substantially penetrate the CNS (see Murakami et al., Comparison of blood-brain barrier permeability in mice abd rats using in situ brain perfusion techniques, Am J Physiol Heart Circ Physiol 279: H1022-1028, 2000, herein incorporated by reference with regard to such teaching). Therefore, Compounds A and B have a good potential to penetrate the brain, but are lower than that of antipyrine. In addition, the average Kᵢₙ values of Compound A, in the absence of Quinidine, was higher than that of Compound B in the absence of Pargyline. Therefore, Compound A likely has a higher intrinsic potential to penetrate the CNS than Compound B.

In the presence of the MAO inhibitor Pargyline, the average brain concentrations and Kᵢₙ values of Compound B were significantly increased relative to the values in the absence of the inhibitor (FIG. 5). Therefore, Compound B's brain penetration appears limited by MAO-mediated degradation. In contrast to the results of Compound B, the co-perfusion of Compound A with the CYP2D6 inhibitor Quinidine did not affect Compound A's brain penetration or Kᵢₙ values (FIG. 6). Therefore, Compound A's brain penetration is not likely to be limited by CYP2D6-mediated degradation.

### Example 4

### Brain-to-Plasma Ratio for Compounds A and B

### Study Objective

The objective of this study was to determine the brain-to-plasma ratio of Compound A and Compound B following oral gavage or intranasal administration in male Sprague-Dawley rats. The influence of an MAO inhibitor, pargyline, on the brain-to-plasma ratio of Compound B was also evaluated.

### Study Design and Methodology

### Materials

Compounds A and B were supplied by Targacept, Inc. (Winston Salem, N.C.). Atenolol and pargyline (MAO inhibitor) were obtained from Sigma-Aldrich (St. Louis, Mo.).

### Animals and Dosing Solutions

Animals used in this study were Sprague-Dawley rats (approximate weight 200400 grams), obtained from Hilltop Lab Animals, Scottdale, Pa. Eight treatment groups of nine rats each were used (see Study Design, Table 6). Upon arrival, the rats were assigned randomly to treatment groups and acclimated for at least 24 hours. The animals were housed up to three per cage and identified by cage labels. A single room was used for this study. The animals were supplied with water and a commercial rodent diet ad libitum. Food was withheld from the animals for a minimum of 12 hours before the study and during the study, and was returned at 4 hours post-dose. Water was supplied ad libitum during the study.

All dosing solutions were prepared in isotonic PBS (phosphate buffered saline) buffer at pH 7.4. Dosing solution concentrations were determined by LC/MS/MS and are shown in Table 7. On the day of the experiment, each rat was administered atenolol at a dose of 10 mg/kg via oral gavage, 30 minutes prior to administration of the test compound. Test compounds (Compound A or Compound B; with or without pargyline) were administered by either oral gavage or intranasally. For intranasal dosing, the animals were anesthetized with CO2 prior to dosing, and the solution was administered in a droplet from a pipette.

TABLE 6
Study Design

| | | | | Test | Test | |
|---|---|---|---|---|---|---|
| | | | Test | Compound | Compound | |
| | | | Compound | Dosing | Dosing | Atenolol¹ |
| | Test | Dosing | Dose | Solution | Volume | Dose |
| Group | Compound | Route | (mg/kg) | (mg/mL) | (mL/kg) | (mg/kg) |
| 1 | Compound B | OG | 10 | 2 | 5 | 10 |
| 2 | Compound B | IN | 0.1 | 2 | 0.05 | 10 |
| 3 | Compound B | IN | 1.0 | 20 | 0.05 | 10 |
| 4 | Compound B + | IN | 0.1 | 2 | 0.05 | 10 |
| | Pargyline² | | | | | |
| 5 | Compound B + | IN | 1.0 | 20 | 0.05 | 10 |
| | Pargyline² | | | | | |
| 6 | Compound A | OG | 5.0 | 2 | 2.5 | 10 |
| 7 | Compound A | IN | 0.1 | 2 | 0.05 | 10 |
| 8 | Compound A | IN | 1.0 | 20 | 0.05 | 10 |

¹Atenolol was administered to each animal via oral gavage at a dosing volume of 1 mL/kg, 30 minutes prior to test compound administration.
²Pargyline (MAO inhibitor) was co-administered at a dosing level of 1 mg/kg.

TABLE 7
Dosing Solutions

| | | Target | Measured Dosing |
|---|---|---|---|
| | | Concentration | Solution Concentration |
| Analyte | Group(s) | (mg/mL) | (mg/mL) |
| Compound B | 1, 2 | 2 | 2.1 |
| | 3 | 20 | 21.6 |
| | 4 | 2 | 2.2 |
| | 5 | 20 | 21.4 |
| Compound A | 6, 7 | 2 | 1.9 |
| | 8 | 20 | 20.0 |
| Atenolol | 1-8 | 10 | 8.6 |

### Sample Collection

For each treatment group, brain and plasma samples were collected at 10, 30 and 60 minutes post-dose. Blood samples were placed in heparinized tubles and spun at 13,000 rpm for 5 minutes. The plasma was placed in polyethylene tubes and frozen (−60 to −80° C.). Brain samples were also placed in chilled tubes and frozen (−60 to −80° C.). Samples remained chilled during subsequent processing.

### Analytical Methodology

### Blank Brain Homogenate

Blank brain homogenate was prepared for use as a diluent for the standard curve and QC preparation. Two whole rat brains were placed in a 50 mL centrifuge tube. To this, 16 mL of 20:80 (v/v) methanol/water was added. The brains were then homogenized using a VirSonic 100 Ultrasonic Cell Disruptor. This procedure was repeated until sufficient homogenate was produced. The products of each homogenization were combined in 50 mL centrifuge tubes and frozen at −80° C. until needed for the analysis.

### Homogenization of Rat Brain Study Samples

Brain samples were thawed and weighed. Sufficient methanol (20% aqueous) was added to each sample to make 4 mL per 1 g of brain tissue, and the mixture was homogenized using sonication with a VirSonic Ultrasonic Cell Disruptor 100 (VirTis). After homogenation, the volume of each sample was recorded and the samples were frozen at −80° C. until analysis.

### Standards and Quality Control Sample Preparation

To determine the concentration of Compound A, Compound B, and atenolol in brain homogenate samples, the standards were prepared in blank rat brain homogenate or pooled rat plasma containing sodium heparin as an anticoagylant, respectively. Plastic tubes were used for all steps. Standards were prepared at concentrations of 1000, 500, 250, 100, 50, 10, 5 and 1 ng/mL by serial dilution. Quality control samples were also prepared at 500, 100, and 5 ng/mL. Compound B was analyzed individually, while Compound A and atenolol were pooled together for simultaneous analysis. Brain standards and quality control samples were treated identically to the test compound samples.

### Sample Extraction

### Compound B

Extraction of brain standards, quality control samples, and study samples for Compound B was carried out on a Tomtec Quadra 96-Model 320 liquid handling system in a 96-well plate format. Brain samples (200 μL) were loaded onto a 96 well plate and then transferred to 400 μL of pure acetonitrile (containing 10 ng/mL nicotine as an internal standard) in a Sirocco Protein Precipitation Plate (Waters Corporation). The resulting suspensions were mixed and then filtered into a clean 96-well collection plate using a vacuum. A 200 μL aliquot of the resulting filtrate was then transferred to plastic HPLC vials for analysis.

Extraction of plasma standards, quality control samples, and study samples for Compound B was carried out on a Tomtec Quadra 96-Model 320 liquid handling system in a 96-well plate format. Plasma samples (50 μL) were loaded onto a 96 well plate and then transferred to 150 μL of pure acetonitrile (containing 10 ng/mL nicotine as an internal standard) in a Sirocco Protein Precipitation Plate (Waters Corporation). The resulting suspensions were mixed and then filtered into a clean 96-well collection plate using a vacuum. A 80 μL aliquot of the resulting filtrate was then transferred to plastic HPLC vials for analysis.

### Compound A and Atenolol

Extraction of brain standards, quality control samples, and study samples for Compound A and atenolol was carried out on a Tomtec Quadra 96-Model 320 liquid handling system in a 96-well plate format. Brain samples (200 μL) were loaded onto a 96 well plate and then transferred to 400 μL of acetonitrile containing internal standard (100 ng/mL Pindolol) in a Sirocco Protein Precipitation Plate (Waters Corporation). The resulting suspensions were mixed and then filtered into a clean 96-well collection plate using a vacuum. The resulting filtrates were then evaporated to dryness under nitrogen at 37° C. The resulting residues were reconstituted with 200 μL of water. The samples were then mixed, centrifuged, and transferred (100 μL) to plastic HPLC vials for analysis.

Extraction of plasma standards, quality control samples, and study samples for Compound A and atenolol was carried out on a Tomtec Quadra 96-Model 320 liquid handling system in a 96-well plate format. Brain samples (50 μL) were loaded onto a 96 well plate and then transferred to 150 μL of acetonitrile containing internal standard (100 ng/mL Pindolol) in a Sirocco Protein Precipitation Plate (Waters Corporation). The resulting suspensions were mixed and then filtered into a clean 96-well collection plate using a vacuum. The resulting filtrates were then evaporated to dryness under nitrogen at 37° C. The resulting residues were reconstituted with 100 μL of water. The samples were then mixed, centrifuged, and transferred (100 μL) to plastic HPLC vials for analysis.

### Method Validation and Extension of Calibration Range in Brain

Methods for determining Compounds A and B in brain homogenate were previously validated as noted hereinabove in the prior study over a range of approximately 200 ng/mL to 1 ng/mL. For this study, this calibration range was extended to 1000 ng/mL, and the precision at this higher calibration level was demonstrated by analysis of six replicates of the high quality control level for each test compound.

Partial validations for Compounds A and B in rat plasma passed all acceptance criteria outlined in the protocol.

### Results

A majority of brain-to-plasma ratios for Compound B could not be determined, because many of the brain samples were below the limit of quantitation of 1 ng/mL. After oral gavage administration, only two brain-to-plasma ratios could be determined, both at the minute time point (average value=0.22). After intranasal administration at 0.1 mg/kg, with or without pargyline, all brain samples were below the limit of quantitation. After intranasal administration at 1 mg/kg without pargyline, only two brain-to-plasma ratios could be determined, both at the 10 minute time point (average value=1.09). After intranasal administration at 1 mg/kg with pargyline, only two brain-to-plasma ratios could be determined at the 10 minute time point (average value=0.89) and one at the 30 minute time point (0.21). Because of the lack of brain-to-plasma values, the effect of added pargyline could not be determined.

The brain-to-plasma ratios for the control compound, atenolol, were low and ranged from 0.144 to 0.025. These low values indicate that the integrity of the blood-brain barrier was maintained throughout the course of the in-life portion of the study.

The results for Compound A are shown in Table 8. After oral gavage administration of Compound A at 5 mg/kg, drug levels were detectable in brain and plasma at all time points. Average brain-to-plasma ratios increased over time, with values of 1.75, 2.37 and 3.38 at 10, 30 and 60 minutes, respectively.

After intranasal administration of Compound A at 0.1 mg/kg, brain-to-plasma ratios could be determined in only three animals, two at the 10 minute time point (average value=2.21) and one at the 30 minute time point (5.52).

After intranasal administration of Compound A at 1 mg/kg, drug levels were detectable in brain and plasma at all time points. Average brain-to-plasma ratios increased over time, with values of 1.58, 4.28 and 6.40 at 10, 30 and 60 minutes, respectively.

These experiments show proof of concept that Compounds A and B can be administered intranasally. Exposures, in both plasma and brain, by both intranasal and oral administration, were much greater for Compound A than for Compound B. Compound A brain-to-plasma ratios were generally greater for intranasal administration, as opposed to oral gavage administration, especially at the later time points. The intranasal administration in test animals was by droplet instead of spray. Greater plasma and brain exposures would be expected from an intranasal dosing that more resembles the typical intranasal spray in terms of the surface area accessed.

TABLE 8
Brain and Plasma Concentrations and Brain-to-Plasma Ratios for Compound A

| | | Dosing | | Brain | Brain | Brain | Brain | Plasma | Brain-to- | | |
|---|---|---|---|---|---|---|---|---|---|---|---|
| | | Route, | Time | Conc. | Wt. | Volume | Conc. | Conc. | Plasma | Mean | |
| Rat # | Group | mg/kg | (min) | (ng/mL) | (g) | (mL) | (ng/g) | (ng/mL) | Ratio | Ratio | SD |
| 1004 | 1 | OG, 10 | 10 | 3.66 | 1.3 | 6.2 | 17.1 | 88.8 | 0.193 | 0.114 | 0.070 |
| 1005 | | OG, 10 | 10 | 2.97 | 1.6 | 7.7 | 14.2 | 235 | 0.060 | | |
| 1006 | | OG, 10 | 10 | 3.58 | 1.7 | 8.0 | 16.6 | 184 | 0.090 | | |
| 1007 | | OG, 10 | 30 | BLOQ | 1.8 | 7.8 | ND | 174 | ND | 0.042 | NC |
| 1008 | | OG, 10 | 30 | 2.04 | 1.5 | 7.2 | 9.6 | 216 | 0.044 | | |
| 1009 | | OG, 10 | 30 | 2.23 | 1.6 | 7.9 | 10.7 | 278 | 0.039 | | |
| 1010 | | OG, 10 | 60 | 1.26 | 1.8 | 8.9 | 6.08 | 158 | 0.038 | 0.032 | 0.007 |
| 1011 | | OG, 10 | 60 | 1.64 | 1.9 | 9.2 | 7.79 | 239 | 0.033 | | |
| 1012 | | OG, 10 | 60 | 1.19 | 2.0 | 9.5 | 5.64 | 234 | 0.024 | | |
| 1013 | 2 | OG, 10 | 10 | 2.18 | 1.7 | 8.1 | 10.4 | 278 | 0.038 | 0.039 | 0.002 |
| 1014 | | OG, 10 | 10 | 2.34 | 1.7 | 8.5 | 11.4 | 275 | 0.041 | | |
| 1015 | | OG, 10 | 10 | 1.04 | 1.8 | 8.6 | 5.04 | 133 | 0.038 | | |
| 1016 | | OG, 10 | 30 | 1.10 | 1.8 | 8.6 | 5.24 | 189 | 0.028 | 0.033 | 0.006 |
| 1017 | | OG, 10 | 30 | 2.11 | 1.8 | 8.6 | 10.1 | 257 | 0.039 | | |
| 1018 | | OG, 10 | 30 | 3.34 | 1.7 | 8.0 | 16.0 | 507 | 0.032 | | |
| 1019 | | OG, 10 | 60 | BLOQ | 1.8 | 8.6 | ND | 134 | ND | 0.029 | NC |
| 1020 | | OG, 10 | 60 | 2.81 | 1.7 | 8.4 | 13.9 | 531 | 0.026 | | |
| 1021 | | OG, 10 | 60 | 1.69 | 1.6 | 7.9 | 8.21 | 265 | 0.031 | | |
| 1022 | 3 | OG, 10 | 10 | 2.31 | 1.7 | 8.1 | 11.0 | 313 | 0.035 | 0.063 | 0.057 |
| 1023 | | OG, 10 | 10 | 1.70 | 1.8 | 8.5 | 8.10 | 327 | 0.025 | | |
| 1024 | | OG, 10 | 10 | 6.77 | 1.8 | 8.3 | 31.9 | 249 | 0.128 | | |
| 1025 | | OG, 10 | 30 | 1.97 | 1.9 | 8.9 | 9.45 | 167 | 0.057 | 0.091 | 0.080 |
| 1026 | | OG, 10 | 30 | 9.95 | 1.9 | 9.0 | 47.6 | 261 | 0.183 | | |
| 1027 | | OG, 10 | 30 | 2.22 | 2.1 | 10.0 | 10.6 | 319 | 0.033 | | |
| 1028 | | OG, 10 | 60 | 3.94 | 1.8 | 8.1 | 18.2 | 638 | 0.028 | 0.027 | 0.001 |
| 1029 | | OG, 10 | 60 | 2.22 | 1.8 | 8.9 | 10.7 | 398 | 0.027 | | |
| 1030 | | OG, 10 | 60 | 2.88 | 1.9 | 9.0 | 13.3 | 523 | 0.026 | | |
| 1031 | 4 | OG, 10 | 10 | BLOQ | 1.8 | 8.4 | ND | 152 | ND | 0.025 | NC |
| 1032 | | OG, 10 | 10 | 1.76 | 1.8 | 8.5 | 8.54 | 340 | 0.025 | | |
| 1033 | | OG, 10 | 10 | 1.34 | 1.8 | 8.5 | 6.45 | 258 | 0.025 | | |
| 1034 | | OG, 10 | 30 | 2.61 | 1.9 | 9.2 | 12.6 | 313 | 0.040 | 0.033 | 0.008 |
| 1035 | | OG, 10 | 30 | 1.52 | 1.9 | 8.9 | 7.20 | 288 | 0.025 | | |
| 1036 | | OG, 10 | 30 | 2.45 | 2.2 | 10.6 | 11.8 | 354 | 0.033 | | |
| 1037 | | OG, 10 | 60 | 2.84 | 1.8 | 8.5 | 13.6 | 353 | 0.039 | 0.036 | 0.003 |
| 1038 | | OG, 10 | 60 | 1.74 | 1.7 | 8.3 | 8.26 | 227 | 0.036 | | |
| 1039 | | OG, 10 | 60 | 2.05 | 1.7 | 8.2 | 10.0 | 314 | 0.032 | | |
| 1040 | 5 | OG, 10 | 10 | 1.17 | 2.0 | 9.3 | 5.56 | 222 | 0.025 | 0.028 | 0.005 |
| 1041 | | OG, 10 | 10 | 1.16 | 1.9 | 8.8 | 5.49 | 164 | 0.033 | | |
| 1042 | | OG, 10 | 10 | 1.27 | 1.8 | 8.6 | 6.06 | 248 | 0.024 | | |
| 1043 | | OG, 10 | 30 | 1.62 | 1.8 | 8.9 | 8.17 | 238 | 0.034 | 0.056 | 0.048 |
| 1044 | | OG, 10 | 30 | 3.10 | 1.9 | 9.1 | 14.9 | 666 | 0.022 | | |
| 1045 | | OG, 10 | 30 | 2.41 | 1.8 | 8.6 | 11.5 | 103 | 0.111 | | |
| 1046 | | OG, 10 | 60 | 3.95 | 1.8 | 8.6 | 18.9 | 509 | 0.037 | 0.034 | 0.003 |
| 1047 | | OG, 10 | 60 | 3.75 | 1.8 | 8.8 | 18.0 | 585 | 0.031 | | |
| 1048 | | OG, 10 | 60 | 1.57 | 2.0 | 9.5 | 7.63 | 222 | 0.034 | | |
| 1049 | 6 | OG, 10 | 10 | 3.62 | 1.9 | 9.4 | 17.5 | 496 | 0.035 | 0.029 | 0.007 |
| 1050 | | OG, 10 | 10 | 1.92 | 1.7 | 8.2 | 9.26 | 439 | 0.021 | | |
| 1051 | | OG, 10 | 10 | 2.14 | 1.8 | 8.8 | 10.3 | 351 | 0.029 | | |
| 1052 | | OG, 10 | 30 | 3.42 | 1.9 | 9.0 | 16.5 | 448 | 0.037 | 0.032 | 0.004 |
| 1053 | | OG, 10 | 30 | 2.94 | 1.8 | 7.8 | 12.8 | 404 | 0.032 | | |
| 1054 | | OG, 10 | 30 | 2.31 | 1.8 | 8.8 | 11.3 | 391 | 0.029 | | |
| 1055 | | OG, 10 | 60 | 2.85 | 1.8 | 8.7 | 13.6 | 364 | 0.037 | 0.036 | 0.008 |
| 1056 | | OG, 10 | 60 | 5.25 | 1.8 | 8.2 | 24.6 | 895 | 0.027 | | |
| 1057 | | OG, 10 | 60 | 4.21 | 1.8 | 8.6 | 20.2 | 473 | 0.043 | | |
| 1058 | 7 | OG, 10 | 10 | 3.96 | 1.7 | 7.6 | 18.1 | 681 | 0.027 | 0.027 | 0.002 |
| 1059 | | OG, 10 | 10 | 3.23 | 1.9 | 8.9 | 15.5 | 538 | 0.029 | | |
| 1060 | | OG, 10 | 10 | 1.44 | 1.9 | 8.8 | 6.83 | 274 | 0.025 | | |
| 1061 | | OG, 10 | 30 | 2.24 | 1.8 | 8.5 | 10.7 | 353 | 0.030 | 0.033 | 0.003 |
| 1062 | | OG, 10 | 30 | 1.76 | 1.8 | 8.4 | 8.42 | 229 | 0.037 | | |
| 1063 | | OG, 10 | 30 | 2.32 | 1.8 | 8.8 | 11.6 | 364 | 0.032 | | |
| 1064 | | OG, 10 | 60 | 2.53 | 1.8 | 8.9 | 12.2 | 488 | 0.025 | 0.030 | 0.004 |
| 1065 | | OG, 10 | 60 | 2.47 | 1.8 | 8.9 | 11.9 | 395 | 0.030 | | |
| 1066 | | OG, 10 | 60 | 1.80 | 1.9 | 9.4 | 8.72 | 259 | 0.034 | | |
| 1067 | 8 | OG, 10 | 10 | 3.81 | 1.8 | 8.8 | 18.3 | 645 | 0.028 | 0.029 | 0.004 |
| 1068 | | OG, 10 | 10 | 2.43 | 1.8 | 8.6 | 11.9 | 356 | 0.034 | | |
| 1069 | | OG, 10 | 10 | 3.94 | 1.8 | 8.7 | 19.0 | 767 | 0.025 | | |
| 1070 | | OG, 10 | 30 | 3.22 | 1.7 | 8.3 | 15.4 | 471 | 0.033 | 0.032 | 0.002 |
| 1071 | | OG, 10 | 30 | 2.09 | 1.6 | 8.0 | 10.2 | 336 | 0.030 | | |
| 1072 | | OG, 10 | 30 | 3.05 | 1.8 | 8.8 | 14.7 | 429 | 0.034 | | |
| 1073 | | OG, 10 | 60 | 3.51 | 1.9 | 9.1 | 16.9 | 473 | 0.036 | 0.040 | 0.004 |
| 1074 | | OG, 10 | 60 | 3.31 | 1.9 | 9.0 | 15.6 | 359 | 0.044 | | |
| 1075 | | OG, 10 | 60 | 2.93 | 1.9 | 9.2 | 14.2 | 345 | 0.041 | | |

BLOQ: Below the Limit Of Quantitation
ND: Not Determined
NC: Not Calculated
* Co-Dosed with Pargyline at 1 mg/kg

Summary of Examples 2 through 4

The foregoing examples establish the ability of Compounds A [(2S)(4E)-N-methyl-5-(3-(5-methoxypyridin)yl)-4-penten-2-amine] and B [(E)-metanicotine] to: (i) cross the respiratory epithelium; (ii) cross the blood-brain barrier; and (iii) establish favorable exposures and brain-to-plasma ratios by the intranasal route of administration.

In laboratory animals, (2S)-(4E)-N-methyl-5-(3-(5-methoxypyridin)yl)-4-penten-2-amine in particular generates very favorable brain-to-plasma ratios via the intranasal route, in a manner which is not compromised by the activity of CYP2D6 (a common drug metabolizing enzyme). By contrast, the attainment of useful (E)-metanicotine exposures by intranasal administration will be aided by co-administration of an MAO inhibitor.

These intranasal examples can be expected to translate well to other mucosal administration methods, including buccal and sublingual routes. Compositions comprising (2S)-4E)-N-methyl-5-(3-(5-methoxypyridin)yl)-4-penten-2-amine, or its salts, and various pharmaceutically acceptable carriers and excipients, described herein, are expected to be effective medicaments when administered by intranasal, buccal, or sublingual means.

### Example 5

### Brain-to-Plasma Ratio for Compound C

### Study Objective

The objective of this study was to determine the brain-to-plasma ratio of Compound C, [(2S-(4E)-N-methyl-5-(3-(5-isopropoxypyridin)yl)-4-penten-2-amine], following oral gavage or intranasal administration in mate Sprague-Dawley rats.

### Study Design and Methodology

### Materials

Compound C, [(2S)-(4E)-N-methyl-5-(3-(5-isopropoxypyridin)yl)-4-penten-2-amine], was supplied by Targacept, Inc. (Winston Salem, N.C.). Atenolol was obtained from Sigma-Aldrich (St. Louis, Mo.).

Animals and Dosing Solutions

Animals used in this study were Sprague-Dawley rats (approximate weight 200-400 grams), obtained from Hilltop Lab Animals, Scottdale, Pa. Three treatment groups of nine rats each were used (see Study Design, Table 9). Upon arrival, the rats were assigned randomly to treatment groups and acclimated for at least 24 hours. The animals were housed up to three per cage and identified by cage labels. A single room was used for this study. The animals were supplied with water and a commercial rodent diet ad libitum. Food was withheld from the animals for a minimum of 12 hours before the study and during the study, and was returned at 4 hours post-dose. Water was supplied ad libitum during the study.

All dosing solutions were prepared in isotonic PBS (phosphate buffered saline) buffer at pH 7.4. Dosing solution concentrations were determined by LC/MS/M. On the day of the experiment, each rat was administered atenolol at a dose of 10 mg/kg via oral gavage, 30 minutes prior to administration of the test compound. Test compound (Compound C) was administered by either oral gavage or intranasally. For intranasal dosing, the animals were anesthetized with CO₂ prior to dosing, and the solution was administered in a 25 μL droplet from a pipette.

TABLE 9
Study Design

| | | | | | | Dosing | | | |
|---|---|---|---|---|---|---|---|---|---|
| | | | Animals | | | Solution | Dosing | | Blood & Brain |
| Treatment | Test | Dosing | N per | Total | Dose | Conc. | Volume | | Sampling Time |
| Group | Compound | Route | timepoint | Animals | mg/kg | mg/mL | mL/kg | Vehicle | Points |
| 1 | Compound C | OG | 3 | 9 | 5 | 1 | 5 | Double | 10, 30, and 60 |
| | | | | | | | | distilled | minutes |
| | | | | | | | | water | |
| 2 | | IN | 3 | 9 | 1 | 8 | 0.125 | Double | 10, 30, and 60 |
| | | | | | | | | distilled | minutes |
| | | | | | | | | water | |
| 3 | | IN | 3 | 9 | 5 | 40 | 0.125 | Double | 10, 30, and 60 |
| | | | | | | | | distilled | minutes |
| | | | | | | | | water | |

Atenolol was administered to each animal via oral gavage at a dosing volume of 1 mL/kg, 30 minutes prior to test compound administration.

For each treatment group, brain and plasma samples were collected at 10, 30 and 60 minutes post-dose. Blood samples were placed in heparinized tubles and spun at 13,000 rpm for 5 minutes. The plasma was placed in polyethylene tubes and frozen (−60 to −80° C.). Brain samples were also placed in chilled tubes and frozen (−60 to −80° C.). Samples remained chilled during subsequent processing.

### Analytical Methodology

Blank Brain Homogenate

Blank brain homogenate was prepared for use as a diluent for the standard curve and QC preparation. Two whole rat brains were placed in a 50 mL centrifuge tube. To this, 16 mL of 20:80 (v/v) methanol/water was added. The brains were then homogenized using a VirSonic 100 Ultrasonic Cell Disruptor. This procedure was repeated until sufficient homogenate was produced. The products of each homogenization were combined in 50 mL centrifuge tubes and frozen at −80° C. until needed for the analysis.

Homogenization of Rat Brain Study Samples

Brain samples were thawed and weighed. Sufficient methanol (20% aqueous) was added to each sample to make 4 mL per 1 g of brain tissue, and the mixture was homogenized using sonication with a VirSonic Ultrasonic Cell Disruptor 100 (VirTis). After homogenation, the volume of each sample was recorded and the samples were frozen at −80° C. until analysis.

Standards and Quality Control Sample Preparation

To determine the concentration of Compound C and atenolol in brain homogenate and plasma samples, the standards were prepared in blank rat brain homogenate or pooled rat plasma containing sodium heparin as an anticoagulant, respectively. Plastic tubes were used for all steps. Standards were prepared at concentrations of 1000, 500, 250, 100, 50, 10, 5, and 1 ng/mL by serial dilution. Quality control samples were also prepared at 500, 100, and 5 ng/mL. Compound C and atenolol were pooled together for simultaneous analysis. Brain standards and quality control samples were treated identically to the test compound samples.

### Sample Extraction

Compound C and Atenolol

Extraction of brain standards, quality control samples, and study samples for Compound C and atenolol was carried out on a Tomtec Quadra 96-Model 320 liquid handling system in a 96-well plate format. Brain samples (200 μL) were loaded onto a 96 well plate and then transferred to 400 μL of acetonitrile containing internal standard (100 ng/mL Pindolol) in a Sirocco Protein Precipitation Plate (Waters Corporation). The resulting suspensions were mixed, then filtered into a clean 96-well collection plate using a vacuum and transferred (100 μL) to plastic HPLC vials for analysis.

Extraction of plasma standards, quality control samples, and study samples for Compound C and atenolol was carried out on a Tomtec Quadra 96-Model 320 liquid handling system in a 96-well plate format. Brain samples (50 μL) were loaded onto a 96 well plate and then transferred to 150 μL of acetonitrile containing internal standard (100 ng/mL Pindolol) in a Sirocco Protein Precipitation Plate (Waters Corporation). The resulting suspensions were mixed, then filtered into a clean 96-well collection plate using a vacuum and transferred (100 μL) to plastic HPLC vials for analysis.

### Method Validation and Extension of Calibration Range in Brain

Method for determining Compound C in brain homogenate was partially validated during the over a range of approximately 1000 ng/mL to 1 ng/mL.

Partial validations for Compound C in rat plasma passed all acceptance criteria outlined in the protocol.

### Results for Compound C

All samples contained residues within the quantifiable range. The results of the analyses are shown in Table 10.

After oral gavage administration of 5 mg/kg, the average plasma levels were 312, 578 and 155 ng/mL at 10, 30 and 60 minutes, respectively. Average brain levels were 225, 1167 and 571 ng/g with brain/plasma ratios of 0.80, 2.2 and 3.5 at 10, 30 and 60 minutes, respectively. After intranasal administration of 5 mg/kg, the average plasma levels were 1463, 421 and 155 ng/mL at 10, 30 and 60 minutes, respectively. Average brain levels were 1753, 1163 and 382 ng/g with brain/plasma ratios of 1.2, 2.8 and 2.4 at 10, 30 and 60 minutes, respectively. After intranasal administration of 1 mg/kg, the average plasma levels were 250, 208 and 103 ng/mL at 10, 30 and 60 minutes, respectively. Average brain levels were 136, 95 and 50 ng/g with brain/plasma ratios of 2.0, 2.8 and 2.4 at 10, 30 and 60 minutes, respectively.

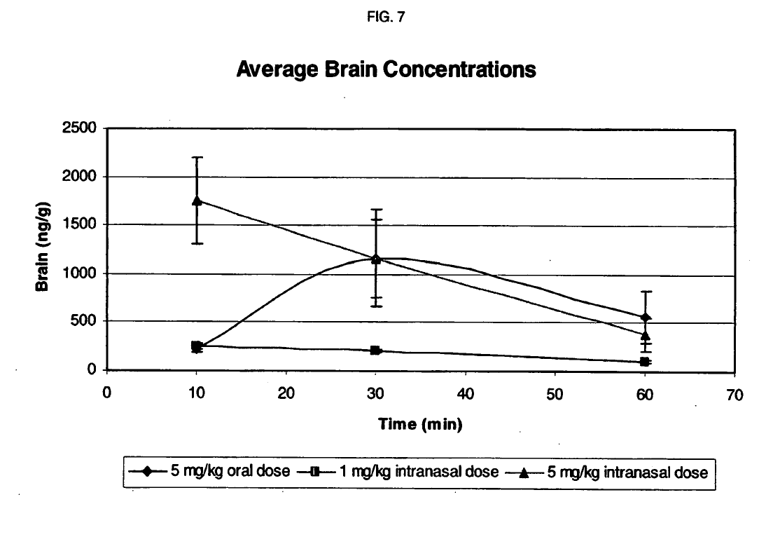

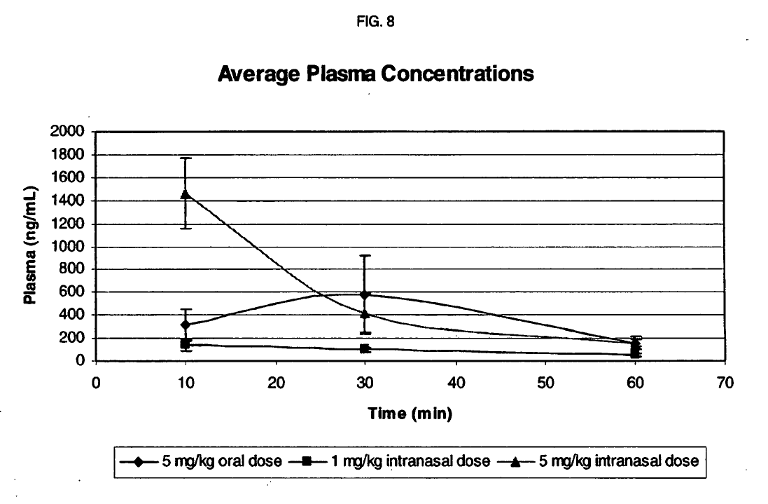

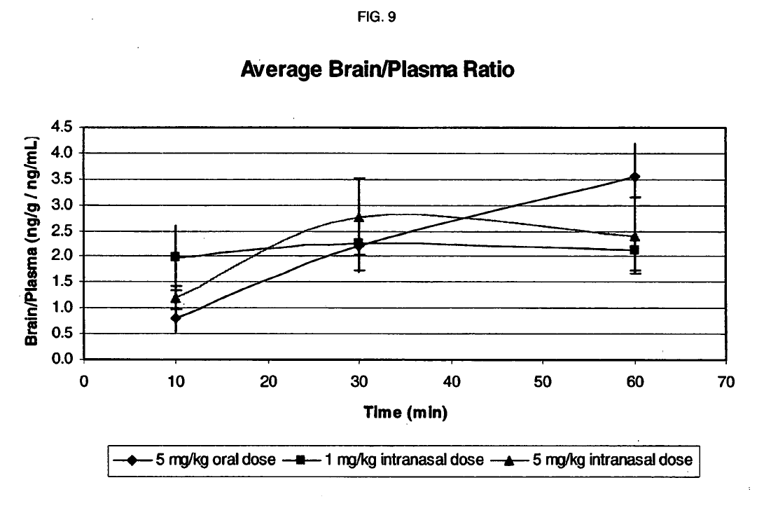

As shown in FIG. 7, at 10 minutes post dose the brain levels for 5 mg/kg doses were 7.8 times higher for intranasal than for oral dosing. A similar trend is seen in FIG. 8 for plasma at 10 minutes post dose, with intranasal being 4.7 times higher than oral. At the and 60 minute timepoints the brain and plasma levels for the two 5 mg/kg dose routes were similar. The brain to plasma ratio for the two 5 mg/kg doses (intranasal/oral) was 1.5, 1.3 and 0.7 at 10, 30 and 60 minutes respectively (FIG. 9).

The brain-to-plasma ratios for the control compound, atenolol, were low and ranged from 0.136 to 0.035. These low values indicate that the integrity of the blood-brain barrier was maintained throughout the course of the in-life portion of the study.

TABLE 10
Brain and Plasma Concentrations and Brain-to-Plasma Ratios for Compound C
5 mg/kg oral dose

| | | | | | | Brain- | | |
|---|---|---|---|---|---|---|---|---|
| | Brain | | Brain | Brain | Plasma | to- | | |
| Time | Conc. | Brain | Volume | Conc. | Conc. | Plasma | Mean | |
| (min) | (ng/mL) | Wt. (g) | (mL) | (ng/g) | (ng/mL) | Ratio | Ratio | SD |
| 10 | 45.4 | 1.74 | 8.7 | 227 | 463 | 0.49 | 0.8 | 0.27 |
| | 37.2 | 1.85 | 9.3 | 186 | 188 | 0.99 | | |
| | 52.5 | 1.69 | 8.5 | 263 | 285 | 0.92 | | |
| 30 | 205 | 1.82 | 9.1 | 1025 | 443 | 2.31 | 2.21 | 0.5 |
| | 171 | 1.84 | 9.2 | 855 | 323 | 2.65 | | |
| | 324 | 1.87 | 9.4 | 1620 | 969 | 1.67 | | |
| 60 | 157 | 1.98 | 9.9 | 785 | 202 | 3.89 | 3.55 | 0.65 |
| | 132 | 1.74 | 8.7 | 660 | 167 | 3.95 | | |
| | 53.7 | 1.88 | 9.4 | 269 | 96 | 2.8 | | |

1 mg/kg intranasal dose

| | | | | | | | Brain- | | |
|---|---|---|---|---|---|---|---|---|---|
| | | Brain | | Brain | Brain | Plasma | to- | | |
| | | Conc. | Brain | Volume | Conc. | Conc. | Plasma | Mean | |
| Rat# | Time (min) | (ng/mL) | Wt. (g) | (mL) | (ng/g) | (ng/mL) | Ratio | Ratio | SD |
| 10 | 10 | 47.6 | 1.8 | 9 | 238 | 88.3 | 2.7 | 1.97 | 0.63 |
| 11 | | 56.8 | 1.77 | 8.9 | 284 | 183 | 1.55 | | |
| 12 | | 45.6 | 1.81 | 9.1 | 228 | 136 | 1.68 | | |
| 13 | 30 | 43.7 | 1.71 | 8.6 | 219 | 78.7 | 2.78 | 2.27 | 0.55 |
| 14 | | 39.9 | 1.74 | 8.7 | 200 | 118 | 1.69 | | |
| 15 | | 40.9 | 1.88 | 9.4 | 205 | 86.8 | 2.36 | | |
| 16 | 60 | 17.7 | 1.88 | 9.4 | 89 | 46 | 1.92 | 2.12 | 0.39 |
| 17 | | 24.6 | 1.8 | 9 | 123 | 66 | 1.86 | | |
| 18 | | 19.4 | 1.8 | 9 | 97 | 37.8 | 2.57 | | |

5 mg/kg intranasal dose

| | | | | | | | Brain- | | |
|---|---|---|---|---|---|---|---|---|---|
| | | Brain | | Brain | Brain | Plasma | to- | | |
| | | Conc. | Brain | Volume | Conc. | Conc. | Plasma | Mean | |
| Rat# | Time (min) | (ng/mL) | Wt. (g) | (mL) | (ng/g) | (ng/mL) | Ratio | Ratio | SD |
| 19 | 10 | 269 | 1.7 | 8.5 | 1345 | 1120 | 1.2 | 1.2 | 0.22 |
| 20 | | 447 | 1.87 | 9.4 | 2235 | 1570 | 1.42 | | |
| 21 | | 336 | 1.81 | 9.1 | 1680 | 1700 | 0.99 | | |
| 22 | 30 | 327 | 1.85 | 9.3 | 1635 | 621 | 2.63 | 2.78 | 0.75 |
| 23 | | 243 | 1.75 | 8.8 | 1215 | 339 | 3.58 | | |
| 24 | | 128 | 1.88 | 9.4 | 640 | 303 | 2.11 | | |
| 25 | 60 | 69 | 1.81 | 9.1 | 345 | 170 | 2.03 | 2.41 | 0.73 |
| 26 | | 114 | 1.86 | 9.3 | 570 | 175 | 3.26 | | |
| 27 | | 46.3 | 1.87 | 9.4 | 232 | 119 | 1.95 | | |

| 5 mg/kg IN/5 mg/kg PO | Brain | Plasma | Ratio |
|---|---|---|---|
| 10 min | 7.8 | 4.7 | 1.5 |
| 30 min | 1.0 | 0.7 | 1.3 |
| 60 min | 0.7 | 1.0 | 0.7 |

### CONCLUSION FOR COMPOUND C

Intranasal absorption into the brain and bloodstream was much faster than oral administration. A higher brain to plasma ratio was achieved through intranasal dosing than oral at 10 and 30 minutes.

These intranasal examples can be expected to translate well to other mucosal administration methods, including buccal and sublingual routes. Compositions comprising (2S-(4E)-N-methyl-5-(3-(5-isopropoxypyridin)yl)-4-penten-2-amine, or its salts, and various pharmaceutically acceptable carriers and excipients, described herein, are expected to be effective medicaments when administered by intranasal, buccal, or sublingual means.

Although specific embodiments of the present invention are illustrated and described in detail, the invention is not limited thereto. The above descriptions are provided as exemplary of the present invention and should not be construed as constituting any limitation of the invention. Rather, modifications will be obvious to those skilled in the art and all modifications that do not depart from the spirit of the invention are intended to be included within the scope of the appended claims.

1. A composition of (2S)-(4E)-N-methyl-5-(3-(5-isopropoxypyridin)yl)-4-penten-2-amine, or a pharmaceutically acceptable salt thereof, along with a pharmaceutically acceptable carrier for intranasal, buccal, or sublingual administration.

2. The composition of claim 1, wherein the compound is the hydroxybenzoic acid salt of (2S)-(4E)-N-methyl-5-[3-(5-isopropoxypyridin)yl]-4-penten-2-amine.

3. The composition of claim 1 further comprising an absorption promoting agent.

4. The composition of claim 1 further comprising one or more excipient, diluent, binder, lubricant, glidant, disintegrant, desensitizing agent, emulsifier, mucosal adhesive, solubilizer, suspension agent, viscosity modifier, ionic tonicity agent, buffer, carrier, surfactant, flavor, or mixture thereof.

5. The composition of claim 1 wherein the composition is a liquid, liquid spray, microspheres, semisolid, gel, or powder.

6. A composition comprising (2S)-(4E)-N-methyl-5-(3-(5-isopropoxypyridin)yl) -4-penten-2-amine, or a pharmaceutically acceptable salt thereof, wherein the composition is a solid dosage form for buccal or sublingual administration that disintegrates in an oral cavity at body temperature and optionally may adhere to the body tissue of the oral cavity.

7. The composition of claim 6 wherein the salt is the hydroxybenzoic acid salt.

8. The composition according to claim 6 further comprising one or more excipient, diluent, binder, lubricant, glidant, disintegrant, desensitizing agent, emulsifier, mucosal adhesive, solubilizer, suspension agent, viscosity modifier, ionic tonicity agent, buffer, carrier, surfactant, flavor, or mixture thereof.

9. The composition according to claim 6 wherein the composition is formulated as a tablet, pill, bioadhesive patch, sponge, film, lozenge, hard candy, wafer, sphere, lollipop, disc-shaped structure, or spray.

10. A method for alleviating pain, comprising administering to a subject in need thereof an effective amount of a composition of claim 1.

11. The method of claim 10, wherein the type of pain is acute pain, chronic pain, neurologic pain, neuropathic pain, female specific pain, post-surgical pain, inflammatory pain, or cancer pain.

12. A method for treating, delaying progression, or preventing the onset of central nervous system disorders, comprising administering to a subject in need thereof an effective amount of a composition of claim 1.

13. The method of claim 12, wherein the central nervous system disorder is associated with an alteration in normal neurotransmitter release.

14. The method of claim 12 wherein the disorder is dyslexia, Parkinsonism, Parkinson's disease, Pick's disease, Huntington's chorea, tardive dyskinesia, hyperkinesia, progressive supranuclear palsy, Creutzfeld-Jakob disease, multiple sclerosis, amyotrophic lateral sclerosis, epilepsy, mania, anxiety, depression, panic disorders, bipolar disorders, generalized anxiety disorder, obsessive compulsive disorder, rage outbursts, Tourette's syndrome, autism, age-associated memory impairment, mild cognitive impairment, presenile dementia, early onset Alzheimer's disease, senile dementia, dementia of the Alzheimer's type, Lewy body dementia, HIV-dementia, vascular dementia, Alzheimer's disease, AIDS dementia complex, attention deficit disorder, attention deficit hyperactivity disorder, schizophrenia, schizophreniform disorder, schizoaffective disorder, or cognitive deficits in schizophrenia.

15. The method of claim 12 wherein the disorder is mild to moderate dementia of the Alzheimer's type, attention deficit disorder, attention deficit hyperactivity disorder, mild cognitive impairment, age associated memory impairment, schizophrenia, or cognitive disorder in schizophrenia.

16-21. (canceled)

22. A method for alleviating pain, comprising administering to a subject in need thereof an effective amount of a composition of claim 6.

23. The method of claim 22 wherein the type of pain is acute pain, chronic pain, neurologic pain, neuropathic pain, female specific pain, post-surgical pain, inflammatory pain, or cancer pain.

24. A method for treating, delaying progression, or preventing the onset of a central nervous system disorder, comprising administering to a subject in need thereof an effective amount of a composition of claim 6.

25. The method of claim 24 wherein the central nervous system disorder is associated with an alteration in normal neurotransmitter release.

26. The method of claim 24 wherein the disorder is dyslexia, Parkinsonism, Parkinson's disease, Pick's disease, Huntington's chorea, tardive dyskinesia, hyperkinesia, progressive supranuclear palsy, Creutzfeld-Jakob disease, multiple sclerosis, amyotrophic lateral sclerosis, epilepsy, mania, anxiety, depression, panic disorders, bipolar disorders, generalized anxiety disorder, obsessive compulsive disorder, rage outbursts, Tourette's syndrome, autism, age-associated memory impairment, mild cognitive impairment, presenile dementia, early onset Alzheimer's disease, senile dementia, dementia of the Alzheimer's type, Lewy body dementia, HIV-dementia, vascular dementia, Alzheimer's disease, AIDS dementia complex, attention deficit disorder, attention deficit hyperactivity disorder, schizophrenia, schizophreniform disorder, schizoaffective disorder, or cognitive deficits in schizophrenia.

27. The method of claim 24 wherein the disorder is mild to moderate dementia of the Alzheimer's type, attention deficit disorder, attention deficit hyperactivity disorder, mild cognitive impairment, age associated memory impairment, schizophrenia, or cognitive disorder in schizophrenia.
